(19) United States
(12) Patent Application Publication
(10) Pub. No.: US 2009/0221484 A1
(43) Pub. Date: Sep. 3, 2009

(54) Use of Factor VII Polypeptides for Neuroprotection

(75) Inventors: Rasmus Rojkjaer, Princeton, NJ (US); Douglas Smith, Concordville/Boothwyn, PA (US)

Correspondence Address: NOVO NORDISK, INC.;INTELLECTUAL PROPERTY DEPARTMENT, 100 COLLEGE ROAD WEST, PRINCETON, NJ, 08540, US

(73) Assignees: Novo Nordisk A/S, Bagsvaerd (DK); The Trustees of The University of Pennsylvania, Philadelphia, PA (US)

(21) Appl. No.: 12/089,415

(22) PCT Filed: Oct. 10, 2006
(86) PCT No.: PCT/US2006/039997

§ 371 Date: Oct. 27, 2008

Related U.S. Application Data
(60) Provisional application No. 60/724,451, filed on Oct. 7, 2005.

(51) Int. Cl. A61K 38/17; A61P 25/28

(52) U.S. Cl. 514/12

(57) ABSTRACT

New methods and compositions for providing neuroprotection in gyrencephalic mammals comprising a neuroprotective dose of Factor Vila or a neuroprotective Factor Vila equivalent are provided.

### CROSS-REFERENCE TO RELATED PATENT APPLICATIONS

This patent application claims the benefit of U.S. Provisional Patent Application No. 60/724,451, filed Oct. 7, 2005, which is incorporated herein by reference in its entirety.

### FIELD OF THE INVENTION

The invention relates to the prevention and treatment of syndromes requiring neuroprotection using Factor VIIa or equivalent polypeptides.

### BACKGROUND OF THE INVENTION

A key feature of the central nervous system (“CNS”) is that differentiated neurons are essentially incapable of regeneration. Permanent loss of function is thus a likely outcome of a sufficiently severe injury or insult to the brain. Accordingly, there is a need for means to protect cells of the central nervous system from death after an injury. Damage to different cell types in the central nervous system, such as, e.g., that which may result from asphyxial, traumatic, toxic, infectious, degenerative, metabolic, ischemic or hypoxic insults, may cause sensory, motor or cognitive deficits. In addition to neurons, glial cells, which are non-neuronal cells in the CNS and are necessary for normal CNS function, may also be affected by various injuries to the brain.

Diseases of the CNS also may cause loss of specific populations of CNS cells. Examples of such diseases include multiple sclerosis (loss of oligodendrocytes as well as myelin); Parkinson's disease (loss of dopaminergic neurons); spinal muscular atrophy (depletion of motor neurons); amyotrophic lateral sclerosis (degeneration of the central pyramidal neurons and spinal motor neurons); Huntington's disease; and epilepsy.

Coagulation factor VIIa plays a key role in the blood coagulation cascade, initiating coagulation in conjunction with tissue factor. Recombinant human factor VIIa (NovoSeven®—Novo Nordisk A/S) and functionally similar polypeptides and polypeptide derivatives (“factor VII polypeptides”) have either been demonstrated or suggested to be effective in the treatment of various bleeding disorders, including in bleeding associated with intracranial hemorrhage and in the context of spinal surgery and stroke (see, e.g., WO 2005123118 (US 20060019894); WO 2005016325 (US 20060205648); WO 2004082708 (US 20060063714); WO 2002055102; and WO 2002062376 (US 20030203845); Alameri et al., Blood Coagul Fibrinolysis. 2005 November; 16(8):573-8; Mayer et al., N Engl J Med 352:777-785, 2005; Weiskopf, European Spine Journal, 13(Supplement 1):S83-S88 (2004); and Lam et al., Ann Pharmacother 39:885-891, 2005).

Compounds that inhibit either tissue factor, such as inactivated factor VII polypeptides, or that directly inhibit factor VIIa, have frequently been demonstrated or suggested to be useful agents in the treatment of neuropathological conditions and related disorders (e.g., stroke) (see, e.g., WO 2004006962; WO 199950254; WO 200305511; WO 2002102380; WO 2003051831; and WO 2003044014). The frequent description of such agents as being useful for neuroprotection may have suggested that Factor VIIa polypeptides would lack neuroprotective properties.

Mindikoglu et al., Digestive Diseases and Sciences, 48(6):1130-1135 (2003) is a case study that mentions a “stabilization” of a “neurological deficit” in a cirrhotic patient suffering from severe coagulopathy and an intraparenchymal hematoma by administration of recombinant human factor VIIa (rhFVIIa). No traumatic brain injury or other neuropathological condition was mentioned in the report. No neurological measurements were reported other than patient observation. The evident focus and purpose of the article is to report the rapid correction of a severe coagulopathy in a cirrhotic male. In this respect, the authors offer no conclusions or suggestions regarding the effects of factor VIIa in respect of neuropathological conditions.

R. A. Bauman et al. (Walter Reed Army Institute of Research) in a non-peer reviewed conference paper entitled “Human Recombinant Factor VIIa is Neuroprotective in a Model of Traumatic Brain Injury and Secondary Hypoxemia” and related poster/abstract (copy currently available at http://stinet.dtic.mil/cgi-bin/GetTRDoc?AD=ADA432989&Location=U2&doc=GetTRDoc.pdf-1-abstract from conference(s) in November 2004 and/or December 2004 available at http://stinet.dtic.mil/oai/oai?verb=getRecord&metadata Prefix=html&identifier=ADA432989) suggest that rhFVIIa “might” be useful in treating hemorrhage and hypotension that result from traumatic brain injury. Bauman et al. subjected rats to fluid percussion injury followed by either visual discrimination testing, Morris Water Maze testing, or visual neurohistological assessment (no efforts were made to quantify neurons). Based on these experiments, the authors speculated that there may be a possibility that rhFVIIa has neuroprotective properties, albeit with some uncertainty (“rhFVIIa was not expected to be neuroprotective and perhaps with the collection of additional data the conclusion will be that it is in fact no neuroprotective”). In addition to the admitted uncertainty concerning the significance of these results, such experiments also offered only limited insights with respect to whether factor VIIa could be a pharmacologically useful neuroprotective agent in humans (e.g., humans suffering from TBI) in that (1) these experiments were performed in rats, a lissencephalic (smooth brain) mammal that poorly responds to rhFVIIa (and therefore requiring remarkably high doses in order to obtain detectable effects); (2) the treated animals received high dosages of mannitol, a known neuroprotective (i.e., due to the high dosage of rhFVIIa formulation needed to obtain an effect); which was not given to untreated animals (3) the experimental controls did not test for the effect of factor VIIa alone; and (4) the work did not actually measure retention of neurons after the injury, and therefore cannot (despite use of the term “neuroprotection”) claim a neuroprotective effect was demonstrated (it is not clear what cell types were implicated in the observed results which were based only on a gross examination of the brain). The failure of numerous compounds that appeared to be promising neuroprotective agents in rodent models, particularly rats, in clinical trials and/or in experiments performed in models that more closely resemble humans have also undermined any meaningful expectation of success in providing neuroprotection in humans based on rodent model data.

Zaaroor et al., Semin Hematol 41:175-176, 2004, reports that intracranial bleeding was arrested in five patients with traumatic brain injury (TBI) after early treatment with recombinant human Factor VIIa (“rhFVIIa”); however the authors did not evaluate whether Factor VIIa promotes neuroprotection in such patients. Dutton et al., J Trauma 57:709-719, 2004, evaluated 20 TBI patients after rhFVIIa therapy and showed improvement in controlling intracranial bleeding, but similarly did not assess neuroprotection in the patients or make any statements regarding the neuroprotective effects of Factor VIIa. Morenski et al., J Neurosurg 98:611-616, 2003, teaches that rhFVIIa can correct the coagulopathy induced by brain injury, but similarly did not assess any neuroprotective effect or suggest that Factor VIIa might be a neuroprotective agent. Moreover, there have been reports concerning intravascular thrombosis in the brain after TBI, raising concerns about whether Factor VIIa is safe in the context of TBI (see, e.g., Stein et al., J Neurosurg 97:1373-1377, 2002 and Stein et al., Neurosurgery 54:687-691; discussion 691, 2004; see also, Roberts et al., Semin Hematol 41:101-108, 2004).

U.S. Pat. No. 6,858,587 (Novo Nordisk A/S) (hereinafter “the '587 patent”) teaches that factor VII polypeptides, including rhFVIIa, can provide neuroprotective effects, particularly in neuropathological conditions primarily associated with or induced by cell apoptosis. Examples of such conditions include amyotrophic lateral sclerosis (ALS), Parkinson's disease (PD), Huntington's disease, Alzheimer's disease (AD), and multiple sclerosis (MS).

There remains a need in the art for methods and compositions for providing neuroprotection in gyrencephalic (convoluted brain) mammals, such as humans, suffering from, or being at substantial risk of developing, additional neuropathological conditions, such as traumatic brain injury. The present invention provides such methods and compositions. These as well as additional advantages and features of the invention will be apparent from the description of the various aspects of the invention provided herein.

### SUMMARY OF THE INVENTION

The invention described herein provides new methods of providing neuroprotection in gyrencephalic mammals comprising administering a neuroprotective dose of Factor VIIa or a neuroprotective Factor VIIa equivalent to the mammal. In this sense, the invention also provides a new use for Factor VIIa or neuroprotective Factor VIIa equivalents for the preparation of medicaments for providing neuroprotection in gyrencephalic mammals. The invention provides other related compositions and methods, selected illustrative examples of which are provided here.

In one aspect, the invention provides a method for providing neuroprotection in a gyrencephalic mammal (e.g., a human) suffering from or at substantial risk of developing a neuropathological condition not primarily induced by apoptosis (e.g., traumatic brain injury) comprising administering to the mammal a neuroprotective-effective dose of Factor VIIa or a neuroprotective Factor VIIa equivalent and assessing the neurological state of the mammal.

In another aspect, the invention provides a method for providing neuroprotection in a gyrencephalic mammal that is suffering from or is at substantial risk of developing a neuropathological condition that manifests as a seizure disorder (e.g., status epilepticus) or neuropsychiatric disorder comprising administering to the mammal a neuroprotective-effective dose of Factor VIIa or a neuroprotective Factor VIIa equivalent (in any case in which a method of treating a neuropathological condition is described herein, the invention provides, in another sense, for the use of Factor VIIa or a neuroprotective Factor VIIa equivalent in the preparation of a medicament for the treatment of that neuropathological disorder, and visa versa).

In still another aspect, the invention provides a method for providing neuroprotection in a gyrencephalic mammal that is suffering from or is at substantial risk of developing a neuropathological condition that manifests as a blinding eye disease (e.g., macular degeneration), comprising administering to the mammal a neuroprotective-effective dose of Factor VIa or a neuroprotective Factor VIIa equivalent.

In yet another aspect, the invention provides a method of providing neuroprotection in a mammal suffering from or being at substantial risk of developing a white matter disease-associated or injury-associated neuropathological condition comprising administering to the mammal a neuroprotective-effective dose of Factor VIIa or a neuroprotective Factor VIIa equivalent. In a particular aspect, the method is limited by the proviso that the neuropathological condition is not Parkinson's disease, Alzheimer's disease, amyotrophic lateral sclerosis (ALS), denervation atrophy, otosclerosis, stroke, dementia, multiple sclerosis, Huntington's disease, or AIDS-associated encephalopathy.

In another exemplary facet, the invention provides a pharmaceutically acceptable composition comprising a combination of a first amount of Factor VIIa or a neuroprotective Factor VIIa equivalent and a second amount of a second therapeutic agent, wherein the first and second amounts together are effective for providing treating a neuropathological condition-related disease in a gyrencephalic mammal. Related kits also are provided.

These exemplary aspects are described further, and additional aspects and feature of the invention are provided, in the description of the invention provided herein.

### BRIEF DESCRIPTION OF THE DRAWINGS

FIGS. 1a-f and 1A-F—Representative MRI images at injury and sacrifice day between two groups of pigs combined TBI model of DAI with focal cortical contusion treated with a recombinant human Factor VIIa (rhFVIIa) composition (NovoSeven®—Novo Nordisk A/S) or control (NovoSeven® excipients only). a, b, c: placebo-treated pig on T2* weighted gradient echo at injury day; d, e, f: placebo-treated pig on T2-weighted TSE at sacrifice day; A, B, C: rFVIIa-treated pig on T2* weighted gradient echo at injury day; D,E,F: rFVIIa-treated pig on T2-weighted TSE at sacrifice day.

FIG. 2—Graph providing a comparison of contusion expansion measurements between rhFVIIa-treated pigs and placebo-treated animals 3 days after the injury.

FIG. 3—Comparison of FVII activity between treatment group and the control group at different time points.

FIG. 4—Comparison of prothrombin time (PT) between treatment group and the control group at different time points.

FIG. 5—Comparison of activated partial thromboplastin time (APTT) between treatment group and the control group at different time points.

FIG. 6a—Graph reflecting severity of pyknotic neurons in the hippocampus in treated and control animals (the average number of pyknotic neurons calculated by semi-quantitative method in two groups—reflecting a neuroprotective effect of rhFVIIa treatment).

FIG. 6b—Graphic illustration of neuronal pyknosis expressed as the percent of pyknotic cells relative to the normal cells in each hippocampal region.

FIG. 7—Graph showing the severity of intravascular coagulation in the brain between two groups on bilateral sides. No significant differences between rFVIIa-treated and placebo-treated animals were discovered on both sides.

FIG. 8—Representative photomicrographs of H&E (A) and Fluorojade (B) stain in three subfields of the hippocampus at three days after injury and treatment (images converted to gray scale for patent publication purposes).

FIG. 9—Graphic representation of the longitudinal diffusion from DTI analysis on rhFVIIa-treated and vehicle-treated animals.

FIG. 10—Graphic representation of the mean diffusion from the DTI analysis.

FIG. 11—Graphic representation of the distribution and severity of axonal injury in vehicle vs. rhFVIIa-treated animals at 3 days post-TBI. Arrows point to regions of the brain in which a “substantial” difference in the severity of axonal damage was seen in the treatment group as compared to the control group.

### DETAILED DESCRIPTION OF THE INVENTION

The present invention stems from the discovery that human Factor VIIa exhibits neuroprotective activity (i.e., provides neuroprotection) in gyrencephalic mammals, particularly in the context of neuropathological disorders where such neuroprotective effects would not be expected based on the earlier teachings of the above-referenced '587 patent.

Neuroprotection refers to the preservation of neural tissue. Neuroprotection typically can be measured by a reduction in death and/or degeneration of neurons and/or a measured reduction in death and/or degeneration of neuron support cells (e.g., astrocytes, Schwann cells, and/or oligodendrocytes) in connection with a neuropathological condition (e.g., neurological injury or disease).

In one aspect, the effect of inventive methods may be primarily, essentially, or entirely limited to reduction of death and/or degeneration of neurons. In a specific aspect of the invention, a measurable effect, and in some aspects the primary effect, of administering Factor VIIa or a neuroprotective Factor VIIa equivalent to the mammal is a measurable reduction in the death of neurons in the mammal. In a particular facet of this aspect, the invention provides a method of promoting neuron sparing in the brain. In an even more particular facet, the invention provides a method for promoting hippocampal neuron sparing.

In another particular aspect, one measurable effect, and in some cases the primary effect of the practice of an inventive method provided herein is a reduction in the degradation of neuronal processes (axons and/or dendrites). In one facet, a measurable effect of the practice of an inventive method, and in some cases the primary effect of the practice of the inventive method, is a reduction in axonal injury, such as a reduction in diffuse axonal injury (DAI). In another aspect, a measurable effect, and in some cases the primary effect, of the practice of inventive method provided herein pronounced is a marked protection of “white matter.”

In another aspect, a measurable result of the practice of an inventive method provided herein is a reduction in death and/or degradation of neuron support cells. Neuron support cells that may be the subject of such sparing or protection may include Schwann cells, oligodendrocytes, and/or astrocytes.

The inventive methods generally can be carried out by administering to such a mammal (e.g., a human patient in need of neuroprotection) human Factor VIIa (all references to “Factor VIIa” herein are to human Factor VIIa unless otherwise indicated), which typically is recombinant, synthetic (a molecule produced by chemical synthesis outside of the context of any cell), or purified (isolated from natural sources) Factor VIIa, or a neuroprotective Factor VII equivalent, in a manner that is effective in providing neuroprotection.

In providing neuroprotection in accordance with any of the various aspects of the invention, Factor VIIa or neuroprotective Factor VIIa equivalent can be delivered to the mammal in any suitable manner that permits promotion of a neuroprotective effect. A manner effective for neuroprotection may comprise, e.g., administering a predetermined amount of Factor VIIa or a neuroprotective Factor VIIa equivalent, and/or utilizing a particular dosage regimen, formulation, mode of administration, combination with other treatments, and the like. Administration of a neuroprotective dose of Factor VIIa or a neuroprotective Factor VIIa equivalent in a pharmaceutically acceptable formulation is a typical mode of providing neuroprotection. In other aspects, Factor VIIa or a neuroprotective Factor VIIa equivalent may be administered by, e.g., delivery and expression of a nucleic acid (DNA, RNA, or other) coding for expression of Factor VIIa or the neuroprotective Factor VIIa equivalent in the mammal (e.g., in the context of a vector, such as DNA or viral vector—for example an attenuated (e.g., replication deficient, non-pathogenic and possibly de-immunized) adeno-associated viral vector, retroviral vector, herpes viral vector, adenoviral vector, pox viral vector, or the like, which may be associated with a tissue-specific or inducible promoter). Examples of relevant constructs and methods are provided in, e.g., Margaritis et al., Semin Hematol. 2006 January; 43(1 Suppl 1):S101-4; US Patent Publication Nos. 20030229036, 20060205036, 20060166915, 20060111282; and Margaritis et al., J Clin Invest. 2004 April; 113(7):1025-31. Ex vivo methods of cell transfer, employment of gene activation techniques, and generally any other suitable method that result in successful administration of a neuroprotective dose of Factor VIIa or a neuroprotective Factor VIIa equivalent to the mammal may also be employed in practicing inventive methods described herein.

The efficacy of employing various methods of the invention in providing neuroprotection in a mammal may be assessed using any suitable one or combination of various well-known neurological assessments and monitoring methods, including, without limitation, conventional imaging methods (e.g., CT, MRI scanning) and/or by evaluation of clinical parameters appropriate for the syndrome being treated. Other suitable methods may be chosen based on minimal invasiveness concerns, sensitivity, etc., which may include, e.g., continuous multiparameter local brain tissue monitoring with microprobes and non-invasive continuous local brain tissue oxygenation monitoring by near infrared spectroscopy (see, e.g., Andrews, Ann NY Acad Sci. 2001 June; 939:101-13), or may be selected based on targeted area (e.g., in the case of optical neurons optical coherence tomography may be used (see, e.g., Sergott, Curr Opin Opthalmol. 2005 December; 16(6):346-50)). More advanced imaging techniques, such as single photon emission computed tomography (SPECT), which can be used to produce an image of the presynaptic dopamine transporter (using a cocaine analog, beta-CIT, as a ligand), and positron emission tomography (PET), which can produce an image of presynaptic dopamine synthesis (using fluorodopa), also may be useful. Additionally useful methods are described elsewhere herein including, e.g., in the Experimental Methods and Data section of this document.

### Indications and Subjects (e.g., Patient Populations):

In one aspect, the invention provides a method for providing neuroprotection in a gyrencephalic mammal suffering from or at substantial risk of developing a neuropathological condition not primarily induced by apoptosis comprising administering to the mammal a neuroprotective-effective dose of Factor VIIa or a neuroprotective Factor VIIa equivalent and assessing the neurological state of the mammal. The invention also relates, in one aspect, to the use of a neuroprotective-effective dose of Factor VIIa or a neuroprotective Factor VIIa equivalent for the manufacture of a medicament for providing neuroprotection in a gyrencephalic mammal suffering from or at substantial risk of developing a neuropathological condition not primarily induced by apoptosis.

A “gyrencephalic mammal” is a mammal having a relatively large brain (e.g., a brain that is at least about 30 grams, but typically is at least about 50 g; commonly at least about 75 g; often at least about 90 g and frequently may be at least about 125 g or more in weight) that is characterized by a highly folded and convoluted cerebral cortex. Examples of such mammals include sheep, pigs, large non-human primates, and humans. The brains of gyrencephalic mammals stand in contrast to the lissencephalic (smooth) brains of small mammals (e.g., rodents). Lissencephalic brains can, for example, tolerate much greater acceleration/deceleration forces than the brains of humans and other gyrencephalic brain mammals. As such, lissencephalic brain mammals typically make poor models for assessing the neuroprotective effects of an agent in gyrencephalic brain mammals, such as humans, particularly in the context of traumatic brain injury (TBI). Unless otherwise indicated, the term “mammal” herein refers to a gyrencephalic mammal.

A mammal in the context of the inventive methods also or alternatively can be characterized as a human Factor VIIa-responsive (or Factor VIIa-sensitive) mammal. A human Factor VIIa-responsive mammal can be characterized as a mammal in which the dose per kg is about 50% or less, such as about 70% or less, or even about 90% or less than the dose of human Factor VIIa required for a similar effect in a rat (e.g., in a pig or dog a dosage of rhFVIIa that is about 50% or less, about 65% or less, or even about 75% or less of the dose used in a rat, on a per kg basis, may result in a similar effect as that in the rat receiving the significantly higher dose).

The phrase “neuropathological condition not primarily induced by apoptosis” simply means that development of the neuropathological condition is attributable primarily to some other factor than programmed cell death (e.g., a neuropathological condition that arises from a physical injury, infection, or exposure to toxic chemicals and/or a condition that arises from cell degeneration, such as diffuse axonal injury). The practitioner will recognize that such neuropathological conditions may be associated with apoptosis, even though apoptosis is not the primary cause of the condition.

The neuropathological treated by the method may be in the central nervous system (CNS), peripheral nervous system (PNS), or both. The neurological condition may be, for example, induced by an injury to the CNS, PNS, or both. In one aspect, the neuropathological condition is induced by an injury to the CNS.

In a particular aspect, the neuropathological condition to be treated is a traumatic brain injury (TBI). TBI is usually the result of a sudden, violent blow to the head. In this respect, leading causes of TBI are vehicle crashes, firearm use, and falls. TBI may be associated with a closed-head injury or an open-head injury (typically associated with penetration of the skull by an object). TBI may also be associated with a deceleration injury (which often is associated with DAI). The TBI may be a concussion. The sTBI may also be associated with chemical/toxic injury (e.g., due to exposure to insecticides, solvents, carbon monoxide poisoning, lead poisoning, or certain drugs such as chemotherapeutic agents), hypoxia, tumors, surgery, or infections (encephalitis or meningitis). TBI in the context of this invention excludes brain injury arising from or associated with stroke or HIV. In one aspect, TBI is associated with an injury caused by differential movement of the brain and skull (which may result in, e.g., axonal shearing, contusion, and/or brain swelling). The TBI may be “mild” TBI (classified by loss of consciousness and/or confusion and disorientation is shorter than 30 minutes, but possibly associated with long-term cognitive problems such as headache, difficulty thinking, memory problems, attention deficits, mood swings and frustration) or severe TBI (which may be associated with, e.g., impairment of limb movement, abnormal speech or language, loss of thinking ability, emotional problems, etc.). The practice of the inventive method may be used to reduce the incidence or severity of such symptoms. As such, the invention also provides for the use of Factor VIIa or a Factor VIIa equivalent in the preparation of medicaments for ameliorating such types of TBI and/or such symptoms of TBI. Unless otherwise indicated all references to “Factor VIIa equivalents” herein are to be understood as referring to neuroprotective Factor VIIa equivalents.

Cerebral contusion is one of the most common focal brain injuries following TBI, which may be associated with neurological deterioration. The volume of hemorrhagic cerebral contusion may be a major factor affecting the morbidity and mortality in TBI patients. In one exemplary aspect, the invention also or alternatively relates to the administration of a neuroprotective dose of Factor VIIa or neuroprotective Factor VIIa equivalent (or the use thereof in the preparation of a medicament) for treatment of TBI, wherein the treatment reduces the volume expansion of intracerebral hematoma and contusion. In a particular aspect, the invention also or alternatively relates to the administration of a neuroprotective dose of Factor VIIa or neuroprotective Factor VIIa equivalent (or the use thereof in the preparation of a medicament) for treatment of TBI associated with a brain contusion (a region of primary neuronal and vascular injury), wherein the administration results in an at least about 3-fold reduction, such as at least about 5-fold reduction, for example an at least about 7-fold reduction, about 8-fold reduction, about 9-fold reduction, or about 10-fold reduction (or more) in the size of the brain contusion after the injury. A brain contusion in the mammal may be caused by direct trauma and/or acceleration (e.g., boxing injury) and/or deceleration (e.g., motor vehicle) injury. An injury also may be generated by one contusion or several contusions. A contusion that may be present, or treated, in the context of practicing some of the inventive methods described herein may classified as a cortical contusion and/or a gliding contusion (which both may be associated with DAI).

In another facet, the invention also or alternatively relates to the administration of a neuroprotective dose of Factor VII or a Factor VII equivalent (or the use thereof in the preparation of a medicament) for the treatment of a TBI in which a significant component of the TBI is DAI and the administration results in a detectable decrease in DAI.

In an additional aspect, the invention also or alternatively relates to the administration of a neuroprotective dose of Factor VII or a Factor VII equivalent (or the use thereof in the preparation of a medicament) for the treatment of TBI, wherein the administration reduces or would reduce the number of pyknotic neurons in the hippocampus after occurrence of the injury by at least about 25% (such as at least about 30%, 35%, 40%, 50%, 60%, 65%, 70%, 75%, or even 80%).

In another exemplary aspect, the neuropathological condition to be treated in the mammal is a spinal injury.

In another exemplary aspect, the neuropathological condition to be treated in the mammal is associated with or secondary to subarachnoid hemorrhage (SAH). In another exemplary aspect, the neuropathological condition to be treated in the mammal is associated with or secondary to intracranial hemorrhage (ICH).

In another exemplary aspect, the neuropathological condition is drug-induced neurodegeneration or toxin-induced neurodegeneration. In a particular facet, Factor VII or a neuroprotective Factor VIIa equivalent can be provided to (or used in the preparation of a medicament for the treatment of) a mammal suffering from or at risk of developing neurodegeneration from exposure to chemotherapeutic or immunosuppressive agents.

In another particular facet, the neuropathological condition is caused by a surgical procedure. For example, in one aspect the invention provides a method for promoting neuroprotection in a mammal suffering from or at risk of developing neurodegeneration secondary to cardiac bypass surgery or other surgery.

In still another exemplary aspect, the neuropathological condition to be treated or prevented is caused by infection (including, without limitation, bacterial meningitis or encephalitis) (but excluding HIV infection), an intracranial tumor, or spinal muscular atrophy.

In another sense, the neuropathological disorder is a neuropathy associated with diabetes. Thus, in another illustrative facet, the invention also or alternatively relates to the administration of a neuroprotective dose of Factor VII or a Factor VII equivalent (or the use thereof in the preparation of a medicament) for the treatment of diabetic neuropathy.

In yet another variation, the neuropathological condition is caused by vitamin deficiency, uremia, anoxia, chronic liver disease, lysosomal storage disease, or fragile X syndrome.

In still another aspect, the invention provides a method for providing neuroprotection in a mammal that is suffering from or is at substantial risk of developing a neuropathological disorder that manifests as a seizure disorder or neuropsychiatric disorder comprising administering to the mammal a neuroprotective-effective dose of Factor VIIa or a neuroprotective Factor VIIa equivalent. In one aspect, the mammal suffers from a seizure disorder. In a particular aspect, the seizure disorder is epilepsy. In an even more particular aspect, the seizure disorder is status epilepticus. In another aspect, the mammal suffers from a neuropsychiatric disorder. For example, the mammal may suffer from, e.g., schizophrenia, depression, autism, and/or mental retardation. The invention similarly provides for the use of a neuroprotective dose of Factor VIIa or a Factor VIIa equivalent in the preparation of a medicament for the treatment of such disorders.

In still another aspect, the invention provides a method for providing neuroprotection in a mammal that is suffering from or is at substantial risk of developing a neuropathological disorder that manifests as a blinding eye disease, comprising administering to the mammal a neuroprotective-effective dose of Factor VIIa or a neuroprotective Factor VIIa equivalent. In a particular exemplary aspect, the blinding eye disease is macular degeneration. In another particular exemplary aspect, the blinding eye disease is retinitis pigmentosa. In still another aspect, the blinding eye disease is glaucoma. The invention similarly provides for the use of a neuroprotective dose of Factor VIIa or a Factor VIIa equivalent in the preparation of a medicament for the treatment of such disorders.

Another useful feature of the invention is embodied in a method of providing neuroprotection in a mammal suffering from or at substantial risk of developing a white matter disease-associated or injury-associated neurological disorder comprising administering to the mammal a neuroprotective-effective dose of Factor VIIa or a neuroprotective Factor VIIa equivalent, with the proviso that the neurological disorder is not Parkinson's disease, Alzheimer's disease, amyotrophic lateral sclerosis (ALS), denervation atrophy, otosclerosis, stroke, dementia, multiple sclerosis, Huntington's disease, or AIDS-associated encephalopathy. One such disease is Alexander's disease. Other white-matter associated disorders that may be treated by employing this aspect of the invention include Canavan's disease; Krabbe's disease; Pelizaeus Merzbacher disease; progressive multifocal leukoencephalopathy (PML); disseminated necrotizing leukoencephalopathy (DNL); acute disseminated encephalomyelitis; Schilder disease; central pontine myelinolysis (CPM); radiation necrosis; Binswanger disease (SAE); Creutzfeldt-Jakob disease; Menkes disease; Vanishing white matter disease (VMW); and other non-specific white matter diseases or injuries. In some aspects, the white matter disorder is characterized by astrocyte degeneration, either primarily or primarily in combination with neuron degeneration. Thus, in one aspect, the invention relates to the treatment of an astrocyte-mediated neurological condition by administering an effective amount of Factor VIIa or a neuroprotective Factor VIIa equivalent. The condition may be a non-specific condition, excluding one or more of the conditions described elsewhere herein (e.g., Parkinson's disease). In such aspects, the method may further comprise the administration of a myelin-promoting agent (e.g., adenosine, progesterone, insulin-like growth factor-I (IGF-I), or interleukin-17 (IL-17)) or application of a myelin-promoting therapy (e.g., electrical impulse therapy). Such agents also may be combined with Factor VIIa or a neuroprotective Factor VIIa equivalent in a pharmaceutically acceptable composition in another facet of the invention.

In some embodiments, the methods and compositions of the invention are applied in the treatment of neuropathological conditions that are not accompanied by clinically significant bleeding. As used herein, “clinically significant bleeding” refers to bleeding that would provoke a treatment response by a medical practitioner or bleeding that would be considered to exacerbate the clinical condition or prognosis of the patient. In some embodiments, the methods and compositions of the present invention are applied to situations in which the Blood-Brain Barrier (BBB) is disrupted.

In other aspects, the neuropathological condition is not associated with intracranial hemorrhage. In a more particular aspect, a mammal treated by a method of the invention may be characterized as being free of any intraparenchymal hematoma. In a more particular aspect yet, inventive methods provided herein may be applied to provide neuroprotection in mammals that can be characterized as being free of an intraparenchymal hematoma caused by a blood vessel disorder, brain tumor, use of blood thinning agent, hemophilia, leukemia, sickle cell anemia, or autoimmune disease. In a still more particular aspect, a mammal to be treated by application of an inventive method may be characterized as lacking any intraparenchymal hematoma that is caused by liver disease. In yet another aspect, the mammal may be characterized as not suffering from cirrhosis. In another aspect, the mammal may be characterized as being free of liver disease.

In still other aspects, a mammal to be treated by application of one of the inventive methods provided herein may be suffering from intracranial hemorrhage or other bleeding condition. In another particular aspect, the mammal may also or alternatively be suffering from an intracranial hematoma. In an even more specific aspect, the mammal may be suffering from an epidural hematoma, a subdural hematoma, and/or an injury-induced intraparenchymal hematoma.

Other neuropathological disorders that may be treated by application of inventive methods provided herein include various dementias (e.g., disease-induced dementias such as are caused by HIV-1 infection); asphyxia, such as, e.g., that associated with fetal or perinatal distress; near-miss drowning, carbon monoxide inhalation, ammonia or other gaseous intoxication; cardiac arrest; cerebral asphyxia associated with coronary bypass surgery; cerebral anoxia or ischemia associated with stroke, hypotensive episodes and hypertensive crises; and cerebral trauma. Metabolic syndromes such as diabetes; chronic liver disease; and infections (e.g., meningitis) may also cause neuronal cell death and therefore be amenable to treatment by application of inventive methods described herein. Mammals suffering from injuries secondary to intracranial tumors also may be treated by inventive methods provided herein.

In one aspect, methods and compositions of the present invention may be advantageously applied to treat any syndrome or neuropathological condition in a mammal, in the absence of treatment, would be accompanied by neurodegeneration (loss of function or structure of neurons). Such neuropathological conditions include, without limitation, stroke (ischemic or hemorrhagic); amyotrophic lateral sclerosis (ALS); Parkinson's Disease (PD); Huntington's Disease (HD); Alzheimer's Disease (AD); spinal muscular atrophy, and multiple sclerosis (MS). Notable aspects of the invention with respect to the treatment of these particular neuropathological conditions may be found in, e.g., the use of neuroprotective factor VIIa derivatives and factor VIIa variants and variant derivatives in such treatment methods; the use of various combination therapies described herein in the treatment of such disorders; and/or the treatment of varieties of such disorders or stages of such disorders that are not primarily characterized by apoptosis.

Another feature of the invention is embodied in a method for treating (or use of Factor VIIa or a Factor VIIa equivalent for preparation of a medicament for treating) Alzheimer's and/or Parkinson's disease arising from traumatic brain injury (TBI). In this aspect, a neuroprotective dose of Factor VIIa or a neuroprotective Factor VIIa equivalent is administered to gyrencephalic mammal (e.g., a human TBI victim) that is or is at substantial risk of developing AD or PD patients develop AD or PD as a result of TBI as opposed to other cause (e.g., age-related apoptosis, apoptosis determined by genetic factors, etc.). Typically, the method is practiced to reduce the likelihood of developing TBI-related PD or AD, reduce the severity of TBI-related PD or AD, delay the onset of TBI-related PD or AD, or a combination thereof, or other prophylactic effect. Practice of such inventive methods may include periodically monitoring the patient for development of symptoms of an early onset PD or AD condition. Such methods may include, for example, monitoring the development of amyloid plaques, generation of amyloid beta, and/or development of signs of early onset for PD or AD in the mammal. In this respect, the invention also provides a method for reducing amyloid plaques in a mammal affected by TBI (e.g., a human TBI patient) by administration of a neuroprotective dose of Factor VIIa or a neuroprotective Factor VIIa equivalent.

Still another aspect of the invention is embodied in reducing the production of amyloid beta and/or amyloid plaques in a gyrencephalic mammal suffering from an axonal injury neuropathological condition comprising administering to the mammal (e.g., a patient suffering from non-TBI-related axonal damage) a neuroprotective dose of Factor VIIa or a neuroprotective Factor VIIa equivalent. In a more particular aspect, the invention provides a method of treating PD or AD in a mammal suffering from axonal injury, such as by reducing likelihood of developing PD or AD.

The invention also encompasses methods and compositions for providing neuroprotection, which are carried out by administering to patient in need of such treatment (i) a first amount of Factor VIIa or a Factor VIIa equivalent in a first treatment regimen and (ii) a second amount of a second therapeutically active agent in a second treatment regimen, wherein the first and second amounts and regimens together comprise a neuroprotective-effective regimen. Non-limiting examples of the second therapeutic agent include: a neurotrophin, a cytokine, an anti-excitatory compound, a calcium channel blocker, a calcium binding protein, an opioid peptide, a barbiturate, an acetylcholinesterase inhibitor, magnesium sulfate, a glutamine receptor antagonist, a steroid (progesterone, dexamethasone), a cannabinoid (dexanabinol), cyclosporin A, anti-Amyloid beta protein, Brain-Derived Neurotrophic Factor, Glial Cell line-derived Neurotrophic Factor, Ciliary Neurotrophic Factor, and neuregulin-1. The invention also encompasses kits that comprise the first and second therapeutic agents and instructions and/or containers for use, mixture, administration, storage, etc.

In another aspect, the invention encompasses methods for providing neuroprotection, which are carried out by (i) administering to a group of patients a neuroprotective effective amount of Factor VIIa or a neuroprotective Factor VIIa equivalent; and (ii) observing an increase in the frequency of one or more indicators of positive clinical outcome among the group of patients relative to the frequency of the indicator(s) that would have been expected in the same group of patients who had not received the Factor VIIa or the neuroprotective Factor VIIa equivalent.

In other aspects, the invention provides methods and compositions for (i) reducing the number of days a patient is hospitalized following trauma or surgery; (ii) methods for improving brain function; (iii) reducing the risk of developing complications of brain dysfunction; and (iv) methods for reducing the risk of progression from brain injury to brain death, all of which comprise administering to a patient in need of such treatment a neuroprotective amount of Factor VIIa or a neuroprotective Factor VIIa equivalent.

In one aspect, a neuroprotective amount of Factor VIIa or a neuroprotective Factor VIIa equivalent is administered in the context of or in association with (before or after) a surgical procedure that is designed to treat a nervous system-related injury, such as TBI.

The practice of inventive methods may be limited to mammals that do not suffer from bleeding disorders, such as bleeding disorders attributable to congenital defects in respect of coagulation factors (e.g., Factor VII, Factor VIII, Factor IX, vWF, etc.) and/or due to presence of inhibitors to such factors. In another aspect, the mammal to be treated also or alternatively does not suffer from a platelet disorder (e.g., Glanzmann's thrombasthenia). In still another aspect, the mammal does not suffer from a persistent bleeding disorder.

Practice of various methods of the invention typically is associated with a step of assessing the neurological state of the mammal after the administration of the neuroprotective dose of Factor VIIa or neuroprotective Factor VIIa equivalent (a neuroprotective dose typically is a dose that is demonstrated to be effective in providing neuroprotection in a number of similar mammals, such as may be determined through pre-clinical or clinical trials, but also may be defined as a dose that is effective for providing neuroprotection in the individual mammal to be treated). Assessment of the neurological state can be done by, e.g., any of the methods described herein for assessing neuroprotection (e.g., MRI, CT, SPECT, etc.). Neurological state also or alternatively may be assessed by neurological functioning of the mammal etc., e.g., by neuropsychological evaluation (in this sense, stabilization or improvement of the neurological state is distinguishable from neuroprotection (defined above)). In one exemplary aspect, assessing the neurological state comprises quantifying neurons (e.g., quantifying hippocampal neuron sparing) and/or assessing proton diffusivity in the white matter of the mammal.

In one aspect, practice of the various methods provided herein may be characterized by the lack of co-administration of any other coagulation factor. In a particular facet, the administration of Factor VIIa or a neuroprotective Factor VIIa equivalent may be characterized by no associated administration of thrombin, Factor Xa, a thrombin inhibitor (e.g., hurudin), and/or a Factor Xa inhibitor (e.g., Tick Anticoagulant Protein). In still another aspect, methods of the invention may be characterized by the lack of administration of any other hemostatic agent in association with administration of the Factor VIIa or neuroprotective Factor VIIa equivalent. In a more particular aspect, the Factor VIIa or neuroprotective Factor VIIa equivalent is the sole active agent that is administered in the practice of the inventive method.

In a another particular aspect, Factor VIIa or neuroprotective Factor VIIa equivalent is administered to the mammal without a high dose of mannitol, such as a dosage that might contribute to a neuroprotective effect. In particular aspects, the amount of mannitol in the Factor VIIa or neuroprotective Factor VIIa equivalent formulation administered to the mammal is limited to less than about 10×, such as less than about 8×, less than about 5×, less than about 3×, or even less than about 2× (on a per kg basis) of the amount of mannitol that would be present in a neuroprotective effective dosage of NovoSeven® when administered to a human patient or less than about 2×, such as less than about 1.5×, the amount of mannitol (on a per kg basis) that would be present in a neuroprotective effective dosage of NovoSeven® when administered to a pig TBI model. For example, in the case of rats, the dosage of NovoSeven® that might be necessary to mimic a neuroprotective effect in humans might likely also comprise a considerably large amount of mannitol (due to the high dosage of hFVIIa that would be necessary in such a model), which may contribute to neuroprotection in a rat.

Unless otherwise stated or clearly contradicted by context, terms such as “treat”, “treating”, and “treatment” herein refer to the delivery of a neuroprotective effective amount of a Factor VIIa or neuroprotective Factor VIIa equivalent (optionally in combination with the administration of one or more additional neuroprotective agents or application of one or more neuroprotective techniques), with the purpose of preventing any symptoms or disease state to develop or with the purpose of easing, ameliorating, or eradicating (curing) such symptoms or disease states already developed. The term “treatment” is thus meant to include prophylactic treatment. However, it will be understood that therapeutic regimens and prophylactic regimens also can be considered separate and independent aspects of the invention (e.g., such regimens may differ in terms of dosage, dosage regimen, etc.). In one aspect, the mammal is suffering from a neuropathological condition, such that the method serves as a therapeutic regimen for the mammal.

In one aspect, the invention provides a method of providing neuroprotection in a mammal that has been determined to be undergoing a neurodegenerative process (e.g., by measurement of neuron and/or glial cell loss) but that does not yet manifest any symptoms of a neurodegenerative disease.

In one aspect, any of the above-described methods may be further characterized by a reduction in the total number of pyknotic neurons in the hippocampus after occurrence of the injury by at least about 25%, such as at least about 50%, such as at least about 60%, or even at least about 70%. In another facet, the inventive methods may be characterized by an at least about 50% (such as at least about 75%, or even about 100%) reduction in the percentage of hippocampal areas characterized by severe pyknosis and/or a reduction of at least about 50% (such as at least about 75%, or even about 100%) in the percentage of hippocampal areas characterized by moderate pyknosis, and/or an up to 50% reduction in the percentage of hippocampal areas characterized by mild pyknosis arising from the neuropathological condition.

One advantageous aspect of the invention is that the administration of Factor VIIa or a neuroprotective Factor VIIa equivalent may be associated with both a detectable (and often therapeutically significant) sparing of hippocampal neurons in a neuropathological condition, e.g., TBI, combined with a sparing of white matter axons.

In another aspect, any of the above-described methods may also or alternatively be further characterized by the administration of Factor VIIa or neuroprotective Factor VIIa equivalent resulting in a detectable reduction of the severity of diffuse axonal injury (DAI) in the mammal. In a particular aspect, any of the various appropriate methods of the invention may result in less, and in some cases substantially less (e.g., by a reduction of about 5% or more, about 10% or more, etc.) swollen axons than in a similar untreated mammal (or population of similar untreated mammals). In another aspect, practice of a method according to the invention may result in a substantial reduction of axonal swelling in an area of the brain in which there is a detectable maintenance in proton diffusion.

In one aspect, any of the above-described inventive methods may optionally comprise a step of determining the amount of hippocampal neuron sparing in the mammal after the injury and administration of the Factor VIIa or neuroprotective Factor VIIa equivalent (and, e.g., repeating administration, increasing dose, etc., until such a therapeutically effective result is shown).

In a particular aspect, the administration of Factor VIIa or neuroprotective Factor VIIa equivalent may be associated with an at least about 25%, such as at least about 50%, such as at least about 70%, or even at least about 80% reduction in hippocampal neuron death.

In still another aspect, practice of the inventive methods may also or alternatively be associated with a reduction in the severity of axonal pathology in one or more areas of the nervous system, such as different areas of the brain. For example, by practicing such methods the severity of axonal pathology may be reduced by at least about 10%, such as at least about 15%, such as at least about 20%, at least about 25%, at least about 35%, or even at least about 50% in, e.g., a frontal lobe area (e.g., the right frontal lobe), a parietal lobe area (e.g., the left parietal lobe), an occipital lobe area (e.g., the right occipital lobe), or basal ganglia (BG) area (e.g., the left basal ganglia).

In yet another aspect, practice of methods provided by the invention may result in a reduction of axonal damage and reduction in proton diffusion in the same white matter tracts. In another aspect, practice of inventive methods may also or alternatively result in a reduced expansion of cerebral contusion and/or reduction in neuronal death (e.g., hippocampal neuronal death).

In still another aspect, the practice of the inventive method may be advantageously characterized by the lack of exacerbation of the severity of intravascular coagulation in the mammal.

In another aspect, the administration of the Factor VIIa or neuroprotective Factor VIIa equivalent is within a dosage that does not detectably promote cerebral thrombosis or embolism. In one facet, the administration of either agent is associated with no statistically significant thrombosis either in the brain and/or in other crucial organs (liver and lung). In a more general sense, a method of the invention may be limited by the lack of any detectable increase in the risk of thrombo-embolic events in the mammal (e.g., the dose of Factor VIIa or Factor VIIa equivalent is high enough to be neuroprotective in the mammal but low enough to avoid such an increase of thrombo-embolic event risk). In a particular aspect, the incidence of thrombo-embolic events associated with the administration of Factor VIIa or neuroprotective Factor VIIa equivalent in the method is about 1% or less. In another aspect, practice of an inventive method may also or alternatively be characterized by the lack of any detectable intravascular thrombi in vital organs of the mammal.

In another aspect, any of the above-described inventive methods may optionally also or alternatively comprise a step of assessing proton diffusivity in the white matter of the mammal after administering the Factor VIIa or neuroprotective Factor VIIa equivalent (and, e.g., repeating administration, increasing dose, etc., until such a therapeutically effective result is shown).

In any of the above-described aspects, the mammal may also be characterized as suffering from an inflammatory condition. Such a condition may be associated with various neuropathological conditions (e.g., AD, PD, or MS).

In another aspect, the administration of Factor VIIa or neuroprotective Factor VIIa equivalent is associated with a reduced expansion of a hemorrhagic cerebral contusion, such as may be associated with TBI. In a particular aspect, expansion of an intracerebral contusion where the inventive method is applied may be about ½ of the expansion that would otherwise occur, such as about ⅓rd, such as about ¼th, ⅕th, or even less than about 1/10th of the expansion that would otherwise occur.

Any or all of the neuroprotective effects of Factor VIIa described above may be distinct from its previously known activity in initiating blood coagulation and/or its effects in respect of apoptosis.

### Factor VIIa and Neuroprotective Factor VIIa Equivalents:

In practicing the present invention, Factor VIIa or any neuroprotective Factor VIIa equivalent may be used. In some embodiments, the Factor VIIa is human Factor VIIa, as disclosed, e.g., in U.S. Pat. No. 4,784,950 (wild-type Factor VII). The term “Factor VII” herein may be used to refer to Factor VII polypeptides in their uncleaved (zymogen) form, as well as those that have been proteolytically processed to yield their respective bioactive forms, which may be designated Factor VIIa. Typically, human Factor VII is cleaved between residues 152 and 153 to yield Factor VIIa.

The previously known activity of Factor VIIa in blood clotting derives from its ability to (i) bind to tissue factor (TF) and (ii) catalyze the proteolytic cleavage of Factor IX or Factor X to produce activated Factor IX or X (Factor IXa or Xa, respectively). The presently discovered neuroprotective activity of Factor VIIa is illustrated in its ability to prevent neuronal cell death, which may require only one of (or none of) TF binding and Factor IX/Factor X proteolytic activity. For use in the present invention, any analogue or derivative of Factor VIIa, or fragment thereof, that exhibits detectable neuroprotective activity may be used, irrespective of whether it exhibits either or both of TF-binding activity or Factor IX/Factor X proteolytic activity.

As used herein, the term “neuroprotective Factor VIIa equivalent” means any variant of human Factor VII (which may be variously referred to as Factor VII variants, Factor VIIa variants, Factor VII analogues, etc.) that exhibit a neuroprotective effect similar to that of human Factor VIIa in a gyrencephalic (and preferably Factor VII-responsive) mammal (e.g., humans) (e.g., by exhibiting at least about 10%, 25%, 35%, 50%, 75%, 90%, 100%, or more than about 100% of the neuroprotective effects of human Factor VIIa); derivatives of human Factor VIIa (e.g., PEGylated forms thereof) that exhibit such neuroprotective effects; naturally-occurring orthologs and paralogs of human Factor VII expressed in man or other species that exhibit such neuroprotective effects; biologically active fragments of hu-man Factor VII, human Factor VII variants, or related orthologs/paralogs; derivatives of Factor VIIa variants and/or orthologs/paralogs that exhibit such neuroprotective effects; fusion proteins comprising domains corresponding to any of the foregoing and that exhibit such neuroprotective effects; and conjugates of any thereof that exhibit such neuroprotective effects. In some embodiments the Factor VIIa or neuroprotective Factor VIIa equivalent is a Factor VII (“FVII”) variant. A Factor VII variant typically exhibits significant structural similarity (e.g., at least about 70%, 75%, 80%, 85%, 90%, 95%, or more sequence identity) to human Factor VII.

Factor VIIa analogues include Factor VII polypeptides that contain one or more sequence alterations relative to wild-type Factor VII. Factor VIIa derivatives include Factor VII polypeptides that are chemically modified relative to human Factor VIIa. Such analogues and derivatives may exhibit different properties relative to human Factor VIIa, including, e.g., improved or decreased stability, phospholipid binding, clot-forming activity, TF-activating activity, and the like.

In one series of embodiments, a neuroprotective Factor VIIa equivalent includes polypeptides that exhibit at least about 10%, 30%, 50%, or 70% of the specific neuroprotective activity of human Factor VIIa. In other embodiments, neuroprotective Factor VIIa equivalents include polypeptides that exhibit at least about 100%, 120%, 150%, 200%, or 300% of the specific neuroprotective activity of Factor VIIa. For purposes of the invention, neuroprotective activity may be quantified by measuring the ability of a preparation to enhance neuronal and/or glial cell survival in an in vivo or ex vivo assay. Non-limiting examples of such assays include: measurement of apoptosis/pyknosis in a model system; analysis of vital receptor function(s); analysis of cell integrity using metabolic markers; and any other conventional assays that measure neuronal cell death.

Neuroprotective activity may also be quantified by measuring one or more biochemical clinical parameters well known to the skilled clinician subsequent to an induced injury or disease. Non-limiting examples of methods for assessing neuronal function include: Measurements of cerebral blood flow; measurements of cerebral oxygen or direct measurements of cerebral metabolic rate (e.g., by MRS, PET, or SPECT scans); assessment of brain integrity using, e.g., MRI, CT, CTA, or MRA; measurement of brain cell electrical function by EEG; measurement of brain function by conventional neurological tests (e.g., Microdialysis, Transcranial Doppler) or cognitive tests.

Non-limiting examples of assays useful for quantifying neuroprotection include those disclosed in: Bishop et al, Mol. Cell. Neurosci. 5: 303-308; Behl et al., 1995. Biochem. Biophys. Res. Commun. 216: 473-482; Green et al, 1996, Neurosci. Lett. 218: 165-168; Goodman et al., 1996, J. Neurochem. 66: 1836-1844; Green et al., 1998, Neuroscience 84: 710; Regan et al., 1997, Brain Res. 764: 133-140, Zaulynov et al., 1999, Cell. Mol. Neurosci. 19: 705-718; Green et al., 1997, J. Neurosci. 17: 511-515; Brinton et al., 1997, Neurochem. Res. 22: 1339-1351; Blum-Degen et al., 1997, Toxicol, Appl. Pharmacol. 152: 49-55; and Simpkins et al., 1997, J. Neurosurg. 87: 724-730.

It will be understood that variants and derivatives of Factor VIIa may be identified using routine experimentation by testing for neuroprotective activity using these or any other suitable methods.

Non-limiting examples of Factor VII variants having substantially the same or increased proteolytic activity compared to recombinant wild type human Factor VIIa include S52A-FVIIa, S60A-FVIIa (Lino et al., Arch. Biochem. Biophys. 352: 182-192, 1998); FVIIa variants exhibiting increased proteolytic stability as disclosed in U.S. Pat. No. 5,580,560; Factor VIIa that has been proteolytically cleaved between residues 290 and 291 or between residues 315 and 316 (Mollerup et al., Biotechnol. Bioeng. 48:501-505, 1995); oxidized forms of Factor VIIa (Kornfelt et al., Arch. Biochem. Biophys. 363:43-54, 1999); FVII variants as disclosed in PCT/DK02/00189 (corresponding to WO 02/077218); and FVII variants exhibiting increased proteolytic stability as disclosed in WO 02/38162 (Scripps Research Institute); FVII variants having a modified Gla-domain and exhibiting an enhanced membrane binding as disclosed in WO 99/20767, U.S. Pat. No. 6,017,882 and U.S. Pat. No. 6,747,003, US patent application 20030100506 (University of Minnesota) and WO 00/66753, US patent applications US 20010018414, US 2004220106, and US 200131005, U.S. Pat. No. 6,762,286 and U.S. Pat. No. 6,693,075 (University of Minnesota); and FVII variants as disclosed in WO 01/58935, U.S. Pat. No. 6,806,063, US patent application 20030096338 (Maxygen ApS), WO 03/93465 (Maxygen ApS), WO 04/029091 (Maxygen ApS), WO 04/083361 (Maxygen ApS), and WO 04/111242 (Maxygen ApS), as well as in WO 04/108763 (Canadian Blood Services) and Factor VIIa lacking the Gla domain, (Nicolaisen et al., FEBS Letts. 317:245-249, 1993).

Further non-limiting examples of FVII variants having increased biological activity compared to wild-type FVIIa include FVII variants as disclosed in WO 01/83725, WO 02/22776, WO 02/077218, WO 03/027147, WO 04/029090, WO 05/075635, and European patent application with application number 05108713.8 (Novo Nordisk A/S), WO 02/38162 (Scripps Research Institute); and FVIIa variants with enhanced activity as disclosed in JP 2001061479 (Chemo-Sero-Therapeutic Res Inst.).

Still further non-limiting examples of additional modifications include: P10Q, K32E, P10Q/K32E, R152E; S344A; L305V; L305V/M306D/D309S; L305I, L305T, F374P, V158T/M298Q, V158D/E296V/M298Q, K337A, M298Q, V158D/M298Q, L305V/K337A, V158D/E296V/M298Q/L305V, V158D/E296V/M298Q/K337A, V158D/E296V/M298Q/L305V/K337A, K157A, E296V, E296V/M298Q, V158D/E296V, V158D/M298K, and S336G, L305V/K337A, L305V/V158D, L305V/E296V, L305V/M298Q, L305V/V158T, L305V/K337A/V158T, L305V/K337A/M298Q, L305V/K337A/E296V, L305V/K337A/V158D, L305V/V158D/M298Q, L305V/V158D/E296V, L305V/V158T/M298Q, L305V/V158T/E296V, L305V/E296V/M298Q, L305V/V158D/E296V/M298Q, L305V/V158T/E296V/M298Q, L305V/V158T/K337A/M298Q, L305V/V158T/E296V/K337A, L305V/V158D/K337A/M298Q, L305V/V158D/E296V/K337A-FVII, L305V/V158D/E296V/M298Q/K337A, L305V/V158T/E296V/M298Q/K337A, S314E/K316H, S314E/K316Q, S314E/L305V, S314E/K337A, S314E/V158D, S314E/E296V, S314E/M298Q, S314E/V158T, K316H/L305V, K316H/K337A, K316H/V158D, K316H/E296V, K316H/M298Q, K316H/V158T, K316Q/L305V, K316Q/K337A, K316Q/V158D, K316Q/E296V, K316Q/M298Q, K316Q/V158T, S314E/L305V/K337A, S314E/L305V/V158D, S314E/L305V/E296V, S314E/L305V/M298Q, 5314E/L305V/V158T, S314E/L305V/K337A/V158T, S314E/L305V/K337A/M298Q, S314E/L305V/K337A/E296V, S314E/L305V/K337A/V158D, S314E/L305V/V158D/M298Q, 5314E/L305V/V158D/E296V, S314E/L305V/V158T/M298Q, S314E/L305V/V158T/E296V, S314E/L305V/E296V/M298Q, S314E/L305V/V158D/E296V/M298Q, S314E/L305V/V158T/E296V/M298Q, S314E/L305V/V158T/K337A/M298Q, S314E/L305V/V158T/E296V/K337A, S314E/L305V/V158D/K337A/M298Q, S314E/L305V/V158D/E296V/K337A, S314E/L305V/V158D/E296V/M298Q/K337A, S314E/L305V/V158T/E296V/M298Q/K337A, K316H/L305V/K337A, K316H/L305V/V158D, K316H/L305V/E296V, K316H/L305V/M298Q, K316H/L305V/V158T, K316H/L305V/K337A/V158T, K316H/L305V/K337A/M298Q, K316H/L305V/K337A/E296V, K316H/L305V/K337A/V158D, K316H/L305V/V158D/M298Q, K316H/L305V/V158D/E296V, K316H/L305V/V158T/M298Q, K316H/L305V/V158T/E296V, K316H/L305V/E296V/M298Q, K316H/L305V/V158D/E296V/M298Q, K316H/L305V/V158T/E296V/M298Q, K316H/L305V/V158T/K337A/M298Q, K316H/L305V/V158T/E296V/K337A, K316H/L305V/V158D/K337A/M298Q, K316H/L305V/V158D/E296V/K337A, K316H/L305V/V158D/E296V/M298Q/K337A, K316H/L305V/V158T/E296V/M298Q/K337A, K316Q/L305V/K337A, K316Q/L305V/V158D, K316Q/L305V/E296V, K316Q/L305V/M298Q, K316Q/L305V/V158T, K316Q/L305V/K337A/V158T, K316Q/L305V/K337A/M298Q, K316Q/L305V/K337A/E296V, K316Q/L305V/K337A/V158D, K316Q/L305V/V158D/M298Q, K316Q/L305V/V158D/E296V, K316Q/L305V/V158T/M298Q, K316Q/L305V/V158T/E296V, K316Q/L305V/E296V/M298Q, K316Q/L305V/V158D/E296V/M298Q, K316Q/L305V/V158T/E296V/M298Q, K316Q/L305V/V158T/K337A/M298Q, K316Q/L305V/V158T/E296V/K337A, K316Q/L305V/V158D/K337A/M298Q, K316Q/L305V/V158D/E296V/K337A, K316Q/L305V/V158D/E296V/M298Q/K337A, K316Q/L305V/V158T/E296V/M298Q/K337A, F374Y/K337A, F374Y/V158D, F374Y/E296V, F374Y/M298Q, F374Y/V158T, F374Y/S314E, F374Y/L305V, F374Y/L305V/K337A, F374Y/L305V/V158D, F374Y/L305V/E296V, F374Y/L305V/M298Q, F374Y/L305V/V158T, F374Y/L305V/S314E, F374Y/K337A/S314E, F374Y/K337A/V158T, F374Y/K337A/M298Q, F374Y/K337A/E296V, F374Y/K337A/V158D, F374Y/V158D/S314E, F374Y/V158D/M298Q, F374Y/V158D/E296V, F374Y/V158T/S314E, F374Y/V158T/M298Q, F374Y/V158T/E296V, F374Y/E296V/S314E, F374Y/S314E/M298Q, F374Y/E296V/M298Q, F374Y/L305V/K337A/V158D, F374Y/L305V/K337A/E296V, F374Y/L305V/K337A/M298Q, F374Y/L305V/K337A/V158T, F374Y/L305V/K337A/S314E, F374Y/L305V/V158D/E296V, F374Y/L305V/V158D/M298Q, F374Y/L305V/V158D/S314E, F374Y/L305V/E296V/M298Q, F374Y/L305V/E296V/V158T, F374Y/L305V/E296V/S314E, F374Y/L305V/M298Q/V158T, F374Y/L305V/M298Q/S314E, F374Y/L305V/V158T/S314E, F374Y/K337A/S314E/V158T, F374Y/K337A/S314E/M298Q, F374Y/K337A/S314E/E296V, F374Y/K337A/S314E/V158D, F374Y/K337A/V158T/M298Q, F374Y/K337A/V158T/E296V, F374Y/K337A/M298Q/E296V, F374Y/K337A/M298Q/V158D, F374Y/K337A/E296V/V158D, F374Y/V158D/S314E/M298Q, F374Y/V158D/S314E/E296V, F374Y/V158D/M298Q/E296V, F374Y/V158T/S314E/E296V, F374Y/V158T/S314E/M298Q, F374Y/V158T/M298Q/E296V, F374Y/E296V/S314E/M298Q, F374Y/L305V/M298Q/K337A/S314E, F374Y/L305V/E296V/K337A/S314E, F374Y/E296V/M298Q/K337A/S314E, F374Y/L305V/E296V/M298Q/K337A, F374Y/L305V/E296V/M298Q/S314E, F374Y/V158D/E296V/M298Q/K337A, F374Y/V158D/E296V/M298Q/S314E, F374Y/L305V/V158D/K337A/S314E, F374Y/V158D/M298Q/K337A/S314E, F374Y/V158D/E296V/K337A/S314E, F374Y/L305V/V158D/E296V/M298Q, F374Y/L305V/V158D/M298Q/K337A, F374Y/L305V/V158D/E296V/K337A, F374Y/L305V/V158D/M298Q/S314E, F374Y/L305V/V158D/E296V/S314E, F374Y/V158T/E296V/M298Q/K337A, F374Y/V158T/E296V/M298Q/S314E, F374Y/L305V/V158T/K337A/S314E, F374Y/V158T/M298Q/K337A/S314E, F374Y/V158T/E296V/K337A/S314E, F374Y/L305V/V158T/E296V/M298Q, F374Y/L305V/V158T/M298Q/K337A, F374Y/L305V/V158T/E296V/K337A, F374Y/L305V/V158T/M298Q/S314E, F374Y/L305V/V158T/E296V/S314E, F374Y/E296V/M298Q/K337A/V158T/S314E, F374Y/V158D/E296V/M298Q/K337A/S314E, F374Y/L305V/V158D/E296V/M298Q/S314E, F374Y/L305V/E296V/M298Q/V158T/S314E, F374Y/L305V/E296V/M298Q/K337A/V158T, F374Y/L305V/E296V/K337A/V158T/S314E, F374Y/L305V/M298Q/K337A/V158T/S314E, F374Y/L305V/V158D/E296V/M298Q/K337A, F374Y/L305V/V158D/E296V/K337A/S314E, F374Y/L305V/V158D/M298Q/K337A/S314E, F374Y/L305V/E296V/M298Q/K337A/V158T/S314E, F374Y/L305V/V158D/E296V/M298Q/K337A/S314E; R152E, S344A; P11Q/K33E, T106N, V253N, R290N/A292T, G291N, R315N/V317T, K143N/R315N/V317T; FVII having substitutions, additions or deletions in the amino acid sequence from T233 to N240; and FVII having substitutions, additions or deletions in the sequence from R304 to C329.

Non-limiting examples of Factor VIIa derivatives include wild-type or analogue Factor VIIa polypeptides to which polyethylene glycol (PEG) polymers, acyl groups, phosphate or sulphate groups, and the like have been attached. Factor VIIa derivatives may also comprise Factor VII polypeptides containing N-linked or O-linked oligosaccharides that have been chemically and/or enzymatically and/genetically modified, such as by alkylation, glycosylation, PEGylation, acylation, ester formation or amide formation or the like, or Factor VIIa (wild type or analogue) polypeptides that have been subjected to proteolytic cleavage. This includes but is not limited to PEGylated human Factor VIIa, cysteine-PEGylated human Factor VIIa and variants thereof. Non-limiting examples of Factor VII derivatives includes GlycoPegylated FVII derivatives as disclosed in WO 03/31464 and US patent applications US 20040043446, US 20040063911, US 20040142856, US 20040137557, and US 20040132640 (Neose Technologies, Inc.); FVII conjugates as disclosed in WO 02/077218, US 20030044908 (Novo Nordisk A/S; WO 01/04287, US patent application 20030165996, WO 01/58935, WO 03/93465 (Maxygen ApS) and WO 02/02764, US patent application 20030211094 (University of Minnesota).

### Preparations and Formulations:

The present invention encompasses therapeutic administration of Factor VIIa or neuroprotective Factor VIa equivalents, which may be achieved using formulations that comprise Factor VIIa preparations. As used herein, a “Factor VII preparation” refers to a plurality of Factor VIIa polypeptides or neuroprotective Factor VIIa equivalent polypeptides that are synthesized or have been separated from the cell in which they were synthesized, whether a cell of origin or a recombinant cell that has been programmed to synthesize Factor VIIa or a Factor VIIa equivalent.

Separation of polypeptides from their cell of origin may be achieved by any method known in the art, including, without limitation, removal of cell culture medium containing the desired product from an adherent cell culture; centrifugation or filtration to remove non-adherent cells; and the like.

Optionally, Factor VII polypeptides may be further purified. Purification may be achieved using any method known in the art, including, without limitation, affinity chromatography, such as, e.g., on an anti-Factor VII antibody column (see, e.g., Wakabayashi et al., J. Biol. Chem. 261:11097, 1986; and Thim et al., Biochem. 27:7785, 1988); hydrophobic interaction chromatography; ion-exchange chromatography; size exclusion chromatography; electrophoretic procedures (e.g., preparative isoelectric focusing (IEF), differential solubility (e.g., ammonium sulfate precipitation), or extraction and the like. See, generally, Scopes, Protein Purification, Springer-Verlag, New York, 1982; and Protein Purification, J.-C. Janson and Lars Ryden, editors, VCH Publishers, New York, 1989. Following purification, the preparation preferably contains less than about 10% by weight, more preferably less than about 5% and most preferably less than about 1%, of non-Factor VII proteins derived from the host cell.

Factor VII and neuroprotective Factor VII equivalent polypeptides may be activated by proteolytic cleavage, using Factor XIIa or other proteases having trypsin-like specificity, such as, e.g., Factor IXa, kallikrein, Factor Xa, and thrombin. See, e.g., Osterud et al., Biochem. 11:2853 (1972); Thomas, U.S. Pat. No. 4,456,591; and Hedner et al., J. Clin. Invest. 71:1836 (1983). Alternatively, Factor VII may be activated by passing it through an ion-exchange chromatography column, such as Mono Q® (Pharmacia) or the like. The resulting activated Factor VII may then be formulated and administered as described below.

Pharmaceutical compositions or formulations for use in the present invention comprise a Factor VIIa preparation in combination with, preferably dissolved in, a pharmaceutically acceptable carrier, preferably an aqueous carrier or diluent. A variety of aqueous carriers may be used, such as water, buffered water, 0.4% saline, 0.3% glycine and the like. The preparations of the invention can also be formulated into liposome preparations or admixed with other excipient compositions that are designed to facilitate delivery or targeting to sites of injury or disease. The compositions may be sterilized by conventional, well-known sterilization techniques. The resulting aqueous solutions may be packaged for use or filtered under aseptic conditions and lyophilized, the lyophilized preparation being combined with a sterile aqueous solution prior to administration.

The compositions may also contain pharmaceutically acceptable auxiliary substances or adjuvants, including, without limitation, pH adjusting and buffering agents and/or tonicity adjusting agents, such as, for example, sodium acetate, sodium lactate, sodium chloride, potassium chloride, calcium chloride, etc.

In one exemplary aspect, the invention provides a pharmaceutically acceptable composition comprising a combination of a first amount of Factor VIIa or a neuroprotective Factor VIIa equivalent and a second amount of a second therapeutic agent, wherein the first and second amounts together are effective for providing treating a neuropathological disorder-related disease in a gyrencephalic mammal. The second therapeutic agent may be, for example, a myelin-promoting agent (examples of which are discussed elsewhere herein). In another facet, the second therapeutic agent is mannitol, hypertonic saline, a barbiturate, a neuromuscular inhibiting agent, or dexanabinol. In still another aspect, the second agent is an anti-inflammatory agent (e.g., an NSAID). In another aspect, the composition might comprise a drug for a blinding eye disorder, such as an anti-angiogenic agent for the treatment of macular degeneration or an anti-glaucoma agent. In still another aspect, the composition might comprise a therapeutic for a neuropsychiatric disorder to be treated, or an anti-seizure medicine. In another aspect, the combination composition comprises a corticosteroid. Typically, the amount of the first and second amounts and regimens together comprises a neuroprotective-effective therapeutic regimen.

Inventive methods described herein also may be applied as an adjunct to employment of other methods designed to promote neuroprotection, such as, e.g., therapeutically inducing hypothermia in a TBI patient, surgically removing blood clots that cause intracranial pressure in a TBI setting, etc. The methods also may be practiced in connection with procedures or techniques targeted to removing a primary cause of a neuropathological condition—such as in association with surgery directed to the removal of a neuropathological disorder-associated intracranial tumor.

Another aspect of the invention is embodied in a kit of parts comprising a first amount of Factor VIIa or a neuroprotective Factor VIIa equivalent in a first unit dose and a second amount of a second therapeutic agent in a second unit dose, wherein the first and second amounts together are effective for providing treating a neuropathological disorder-related disease in a gyrencephalic mammal.

Still another aspect of the invention is embodied in a kit of parts for providing neuroprotection in a gyrencephalic brain mammal that is suffering from or is at substantial risk of developing a neuropathological condition primarily induced by an injury to the central nervous system (CNS), comprising (i) a medicament comprising a neuroprotective-effective dose of Factor VIIa or a neuroprotective Factor VIIa equivalent; and (ii) instructions for Use describing an administration scheme, for example, a. a first dose containing at least about 20 μg/kg, such as at least about 40 μg/kg, such as at least about 80 μg/kg, such as at least about 100 μg/kg of said Factor VIIa or a neuroprotective Factor VIIa equivalent, should be administered at the start of treatment; b. optionally, a second dose containing at least about 40 μg/kg, such as at least about 80 μg/kg Factor VIIa or a neuroprotective Factor VIIa equivalent should be administered one to 24 hours after the start of treatment. In another exemplary aspect, the invention provides such a kit wherein the instructions for use further describes that an optional third dose containing at least about 20 μg/kg, such as at least about 40 μg/kg, such as at least about 80 μg/kg Factor VIIa polypeptide having increased activity compared to wild-type Factor VIIa may be administered to said subject at least about one hour after the start of the second treatment.

### Treatment Regimens:

In practicing the present invention, Factor VIIa or a neuroprotective Factor VIIa equivalent may be administered to a mammal, e.g., a human patient, as a single dose comprising a single-dose-effective amount for providing neuroprotection, or in a staged series of doses which together comprise an effective amount for preventing or treating complications. A neuroprotective effective amount of Factor VIIa or the neuroprotective Factor VIIa equivalent refers to the amount of Factor VIIa or Factor VIIa equivalent which, when administered in a single dose or in the aggregate of multiple doses, or as part of any other type of defined treatment regimen, produces a measurable statistical improvement in outcome, as evidenced by at least one clinical parameter associated with the syndrome or condition being treated.

Administration of a single dose refers to administration of an entire dose of Factor VIIa or the Factor VIIa equivalent as a slow bolus over a period of less than about 5 minutes. Typically, a neuroprotective effective amount comprises at least about 40 μg/kg human Factor VIIa or a corresponding amount of a Factor VIIa equivalent, such as, at least about 50 μg/kg, 75 μg/kg, or 90 μg/kg, or at least about 160 μg/kg Factor VIIa. When neuroprotective Factor VIIa equivalents are administered, the corresponding effective amount may be determined by comparing the neuroprotective activity of the Factor VIIa equivalent with that of Factor VIIa and adjusting the amount to be administered proportionately to the predetermined effective dose of Factor VIIa.

It will be understood that the effective amount of Factor VIIa or Factor VIIa equivalent, as well as the overall dosage regimen, may vary according to the particular disease or situation and patient's presenting status, which, in turn, may be reflected in one or more conventionally known clinical parameters. An effective amount also or alternatively may be determined by those of ordinary skill in the art by routine experimentation, e.g., by constructing a matrix of values and testing different points in the matrix.

In the practice of inventive methods described herein, Factor VIIa or a neuroprotective Factor VIIa equivalent may be administered by any effective route, including, without limitation, intravenous, intracerebral (e.g., by injection into he cisterna magna) intramuscular, subcutaneous, transdermal, mucosal (e.g., intranasally), and pulmonary routes of administration. Preferably, administration of Factor VIIa or a neuroprotective Factor VIIa equivalent polypeptide or polypeptide derivative is by an intravenous route.

### Combination Treatments:

As noted above, the present invention encompasses combined administration of an additional agent in concert with Factor VIIa or a Factor VIIa equivalent. In some embodiments, the additional agent comprises a coagulant, including, without limitation, a coagulation factor such as, e.g., Factor VIII, Factor IX, Factor V, Factor XI, or Factor XIII; or an inhibitor of the fibrinolytic system, such as, e.g., PAI-1, aprotinin, ε-aminocaproic acid or tranexamic acid. In other embodiments, the additional agent comprises a second neuroprotective agent, including, without limitation, a neurotrophin, a cytokine, an antiexcitatory compound, a calcium channel blocker, a calcium binding protein, an opioid peptide, a barbiturate, an acetylcholinesterase inhibitor, magnesium sulfate, a glutamine receptor antagonist, a steroid (progesterone, dexamethasone), a cannabinoid (dexanabinol), cyclosporin A, Anti-Abeta (anti-Amyloid beta protein, Golde et al., Brain Pathol. 2005 15(1):84-7), BDNF (Brain-Derived Neurotrophic Factor, Sharma et al., Ann N Y Acad. Sci. 2005; 1053:407-21), GDNF (glial cell line-derived neurotrophic factor, McBride et al., Prog Brain Res. 2002; 138:421-32), CNTF (ciliary neurotrophic factor, Holm et al., J. Neurochem. 2002 August; 82(3):495-503), and neuregulin-1 (Xu et al., J Cereb Blood Flow Metab. 2005).

It will be understood that, in embodiments comprising administration of combinations of Factor VIIa with other agents, the dosage of Factor VIIa or Factor VIIa equivalent may on its own comprise an effective amount and additional agent(s) may further augment the therapeutic benefit to the patient. Alternatively, the combination of Factor VIIa or equivalent and the second agent may together comprise a neuroprotective effective amount. It will also be understood that effective amounts may be defined in the context of particular treatment regimens, including, e.g., timing and number of administrations, modes of administrations, formulations, etc.

The present invention also provides kits suitable for use in neuroprotective treatments. The kits comprise: (ii) a pharmaceutical preparation comprising Factor VIIa or a neuroprotective Factor VIIa equivalent; and (ii) instructions for use. In some embodiments, the kit may also comprise a second therapeutic agent as described above.

### Other Aspects of the Invention

The invention further provides method of promoting the sale and/or use of Factor VIIa or a neuroprotective Factor VIIa equivalent (or a particular formulation comprising the same—such as, e.g., NovoSeven®), or other related compound or composition described herein, comprising distributing information (e.g., by printed materials that are handed out, mailed, etc.; by advertising signage; by television programs and advertisements; by radio programs and advertisements; by internet site postings; by email; by telemarketing; by door-to-door or person-to-person marketing; by funding and/or hosting conferences, panels, forums, etc., by employing and/or contracting for the services of salespeople and/or medical/scientific liaisons, by funding and/or hosting scientific research and publications related to such uses, etc.) related to the use of the compound or composition in the prevention or treatment of any condition or combination of conditions recited in any of the foregoing aspects or described elsewhere herein to any persons or entities of potential interest (e.g., pharmaceutical chains, formulary managers, insurance companies, HMOs, hospitals and hospital chains, other health care companies, pharmacy benefit managers, patients suffering from or potential patients at substantial risk of developing one of the target neuropathological conditions described herein, primary care physicians, nurses, doctors of pharmacy, and/or key opinion leaders).

In still another aspect, the invention provides a method for identifying a neuroprotective Factor VIIa equivalent use for therapy in humans, which comprises administering a candidate Factor VIIa equivalent to a suitable gyrencephalic mammal model (e.g., a TBI model such as the pig TBI models described in detail herein) and assessing the neuroprotective effect of the candidate Factor VIIa equivalent so as to determine if the candidate is neuroprotective in the model.

### Experimental Data and Methods

The following examples are intended as non-limiting illustrations of particular aspects of the present invention and related principles and methods.

### Neuroprotective Effect of FVIIa in a Pig Model for TBI

A combined TBI model of DAI with focal cortical contusion in pigs was developed to evaluate the effects of early post-injury treatment with rhFVIIa. Both safety and potential efficacy of rhFVIIa therapy in this combination TBI model were evaluated. The initial major goal of this study was to determine if rhFVIIa would reduce the expansion of hemorrhagic cerebral contusion after TBI. A secondary goal was to evaluate the risk of intravascular thrombosis in this setting. In addition, based on the potential risk of increased intravascular thrombosis after rhFVIIa administration, hippocampal pathology and diffuse axonal injury (DAI) were evaluated to determine the relationship between these pathological changes and intravascular thrombosis in the brain.

### Materials and Methods

### Animal Preparation

Ten miniature female adult swine (6-7 months of age, Hormel strain), each weighing 22-25 kg, were used for this study (5 rhFVIIa-treated animals and 5 vehicle-treated controls). The animals were housed in a facility that was accredited by the Association for Assessment and Accreditation of Laboratory Animal Care International. The protocol was approved by the Animal Care and Use Committee of the University of Pennsylvania, and all animals received care in strict compliance with the “Guide for the Care and Use of Laboratory Animals”. Animals were fasted 12 hours prior to the surgical procedure. After sedation with injection of midazolam (400-600 mg/kg), anesthesia was induced with 4-5% isoflurane via snout mask until they reached a plane of surgical anesthesia. Animals were endotracheally intubated and maintained in general anesthesia with isoflurane (1.5-2%). During the procedure, a pulse oximeter was placed on the ear to monitor physiological changes as reflected in oxygen or heart rate.

### Induction of Traumatic Brain Injury

Diffuse axonal injury and focal cortical contusion were induced by our double injury model in the pig. DAI was induced via non-impact rotational acceleration by a sophisticated HYGE pneumatic actuator that was previously described in detail (Smith et al., J Neuropathol Exp Neurol 56:822-834, 1997; Smith et al., J Neurosurg 93:315-322, 2000). Briefly, the animals' heads were secured to a padded snout clamp, which, in turn, is mounted to the linkage assembly of the HYGE device. The linkage assemblies were adjusted to produce pure impulsive head rotation 110° in the coronal plane in 20 msec. This injury level has been characterized to induce extensive DAI through both cerebral hemispheres. Following acceleration, the animals' head was released from the clamp.

Cortical contusion was superimposed following this initial injury. This second injury, termed dynamic cortical deformation (DCD), may produce focal cerebral contusion characterized as focal hemorrhagic lesions. In brief, a straight midline incision was made in the scalp to allow access to drill an 8 mm burr hole through the skull 1 cm lateral from the sagittal suture, centered between lambda and bregma. After opening the dura, a rigid plastic tube (8 mm external diameter, 6 mm internal diameter) was cemented into the burr hole with bone wax. This tube was coupled to a vacuum pulse generator via a flexible tube. This device is a simple solenoid driven machine constructed for this experiment. Cerebral contusion injury was induced by applying a 1 atmosphere negative pressure for 2 seconds through the burr hole. After contusion, the incision was sutured and dressed, and animals were transported to the MRI facility for scanning.

### Drug Administration and Blood Collection

Bilateral femoral veins and arteries were exposed surgically before rotational injury. At 5 min after cerebral contusion, animals received an IV bolus via the femoral vein of either rhFVIIa (720 μg/kg, NovoSeven; Novo Nordisk A/S, Bagsvaerd, Denmark) or a vehicle-control (excipients only). Blood samples were collected from the femoral artery at four time points: pre-injury, 5 min following administration of drug or vehicle (acute time point), 2 hrs post-injury, and 72 hrs following injury (prior to sacrifice). All samples were collected through an 18G IV catheter.

### Magnetic Resonance Imaging (MRI) and Analysis of Cortical Contusion Expansion

Brain imaging was performed using a 1.5T Siemens MRI system at 20 min following brain trauma, and at three days post-injury. The animals were maintained in general anesthesia during MRI examinations. Pigs were placed in the prone position, and 13-cm surface coil was positioned on the head. T1-weighted images (TR/TE1900/5.6 ms, slice thickness 3 mm), T2-weighted TSE images (TR/TE 2500/81 ms, slice thickness 3 mm), and T2* weighted gradient echo images (TR/TE 4480/46, slice thickness 5 mm) were acquired. T2* weighted gradient echo is the most sensitive sequence to detect hyperacute hemorrhage in the brain. This sequence was selected to identify and measure the contusion volume on the injury day. On the sacrifice day, by contrast, hemorrhagic contusions were best identified by T2 weighted TSE. The T2-weighted TSE images were characterized by a hypo-intense contusion surrounded by hyper-intense edema. Thus, T2 weighted TSE images were used for the measurement of contusion volume on the sacrifice day (i.e., 3 days post injury). The volume of cerebral contusion was evaluated by a blinded neuroradiologist using Leonardo workstation imaging software (Siemens, Germany). The expansion of the cortical contusion was expressed as a ratio of lesion volume on injury day to sacrifice day divided by the lesion volume on the injury day.

### Diffusion Tensor Imaging—Image Processing

Diffusion Tensor Imaging (DTI) data were analyzed with DTI Studio (available from Johns Hopkins Medical Institute—see, e.g., http://lbam.med.jhmi.edu/). Regions of interest (ROIs) were placed in the frontal and occipital subcortical and deep white matter, as well as within the descending white matter tracts at level of the internal capsule. For each ROI mean values for FA, mean diffusivity, E1, E2 and E3 were obtained. Longitudinal diffusion (parallel to white matter tracts) was presumed to be equivalent to E1 and transverse diffusion (perpendicular to white matter tracts) was considered the average of E2 and E3.

### Coagulation Parameters Analysis

Blood samples were assayed for FVII:C, FVIIAg level, prothrombin time (PT), and activated partial thromboplastin time (APTT). FVII Coagulant was measured and the FVII antigen level was determined using a FVII EIA (Dako A/S, Denmark). PT was performed with the thromboplastin C (Dade Behring) reagent that gives longer clotting times. APTT was performed with APTT-LA® (Stago, France) that is used for detection of lupus anticoagulant since this reagent gives longer clotting times and theoretically is more sensitive to changes in coagulation state.

### Tissue Preparation

Three days after TBI, all animals were sacrificed by an intravenous overdose of pentobarbital (150 mg/kg), and were transcardially perfused with 4 L saline followed by 8 L 4% paraformaldehyde. The brain, lungs and livers were removed, post-fixed in 4% paraformaldehyde for 2 hrs, and stored in phosphate buffer solution. The brains were blocked into 5 mm coronal sections for gross examination and photography. Tissue blocks from some animals were cryoprotected in sucrose, and a series of 40-μm frozen sections were cut from the front face of each block and mounted on slides for microscopy. Representative slides from each block were stained with haemotoxylin-eosin and cresyl violet. Blocks from lungs and livers were processed for paraffin embedding in an automated tissue processor. Serial paraffinized sections 6 μm thick were cut, mounted on slides prepared for staining.

### The Volumetric Analysis of Cerebral Contusion by Gross Pathological Examination

The volume of cerebral contusion was measured post-mortem in all animals to verify the MRI findings. The contusion region in the fixed brain was blocked into 5 mm coronal sections. Each block containing the contusion lesion was photographed and transferred to a personal computer for analysis. One slice from each block was also selected for H&E stain, and microbleedings which were not visible in the gross examination were detected and photographed under the microscope. A neuropathologist, masked to dose assignment, identified lesion boundaries in the specimens, defined as areas of hemorrhage and necrosis with standard imaging software (Image), NIH), and the volume of microbleedings were also evaluated with the same software. The total volume of cerebral contusion and microbleedings was recorded and compared with the findings from the final MR scan.

### Immunohistochemical Methods

To detect axonal pathology, immunohistochemistry was performed on frozen sections by using the avidin-biotin immunoperoxidase complex method. Primary monoclonal antibody NF52, targeting the 200-kD neurofilament subunits (Sigma, 1:400), was used. The brain sections were incubated with primary antibody overnight at 4° C. and then incubated at room temperature for 1 hour each with appropriate secondary antibodies and antibody complexes were detected using avidin-biotin peroxidase(ABC) histochemistry (Vector Labs). Peroxidase activity was revealed using 0.025% DAB, 300 mg imidazole, and 0.25% hydrogen peroxide for 10 minutes.

In order to detect the intravascular coagulation, an antibody was used to recognize free antithrombin III and antithrombin III contained in antithrombin III-protease complexes (rabbit polyclonal antibody, 1:400; Dako, Carpinteria, Calif.). Brain sections were incubated overnight at 4° C. with primary antibody and then incubated with fluorescein isothiocyanate anti-rabbit immunoglobulin G secondary antibody.

### Histopathological Analysis

For assessment of hippocampal neuron pathology, 5 adjacent brain sections (40 μm) were taken from the mid-dorsal hippocampus in the coronal plane. The pyramidal cell layer was examined for pyknotic neurons (darkly staining shrunken profiles) in three hippocampal subfields (CA1, CA2, and CA3) for each animal. An ordinal scoring system was used to describe the average extent of neuronal pyknosis in each subfield as; 0=no neuronal pyknosis, 1+=mild pyknosis affecting <5 neurons, 2+=moderate pyknosis affecting 5-15 neurons, 3+=severe pyknosis affecting >15 neurons. A semi-quantitative method (as described in Schrieber et al., J Neuropathol Exp Neurol 58:153-164, 1999) was used to evaluate the severity of hippocampal neuron pyknosis in both groups.

The severity and distribution of axonal damage was evaluated according to previously developed sector scoring techniques (Smith et al., 3 Neuropathol Exp Neurol 56:822-834, 1997; Smith et al., J Neurosurg 93:315-322, 2000). Brain regions including frontal, parietal, temporal, occipital lobes, basal ganglion and brain stem were examined. Each coronal section was subdivided into a population of microscopic fields across each coronal section, each containing an area of 1.2 mm². Scoring of the density of axonal pathology/field was determined as: 1-5 swollen axonal profiles within a 1.2 mm² field was designated as mild axon injury, 6-15 profiles as moderate axon injury, and over 15 profiles as severe axon injury. The population of 1.2 mm² fields (mild, moderate, and severe) was tallied in each section, and the average number of mild, moderate, and severely injured fields was recorded for each brain region.

The extent and severity of intravascular thrombosis in the brain was analyzed in a similar fashion as performed with DAI analysis.

### Statistical Analysis

All the data was screened for normality. All statistical analyses were carried out using exact non-parametric tests. The methods used are valid for small samples in which the data cannot be assumed to have a normal distribution. The Type I error rate was 0.05 for all hypothesis tests. For cerebral contusion volume, exact Wilcoxon rank-sum tests were used to determine whether there were differences between (i) initial brain volume at time of injury, and (ii) percent expansion of brain volume at time of sacrifice. Kendall's tau test was used to estimate the association between histopathological and MRI volumes of brain samples.

### Results

### Cerebral Contusion Expansion Analysis on MRI

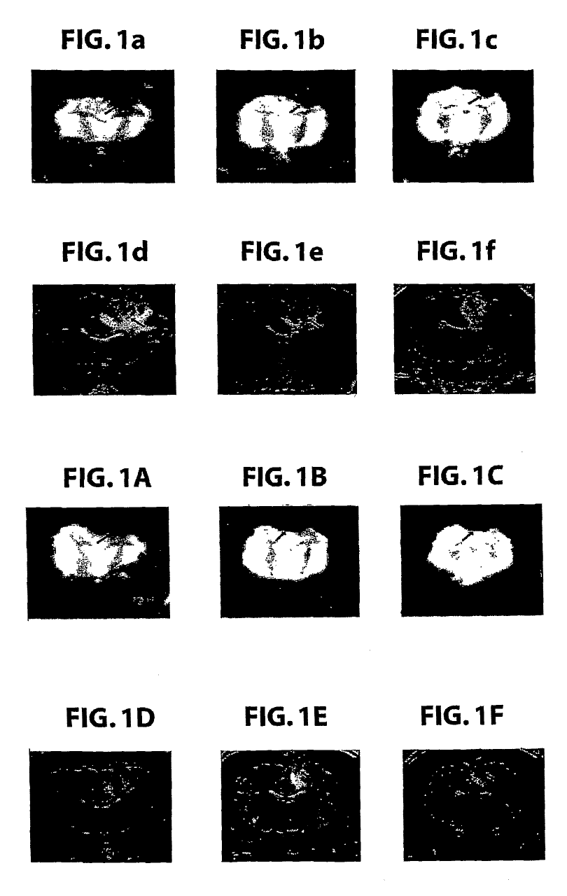

The criteria for selecting MRI sequences in this study to detect the hemorrhagic cerebral contusion is based on the development of cerebral contusions over three days after injury, which can be reliably detected by different MRI sequences. In the hyperacute stage (<12 h post injury), the lesions were mainly composed of intraparenchymal hemorrhages, in which T2* weighted gradient echo (GE) is reported to be the most sensitive MRI sequence to identify acute blood in the brain. In the current study, T2* weighted (GE) sequence detected the intracerebral hemorrhages in all animals, as easily identified hypointense areas (FIG. 1). In contrast, three days after injury, the components of the hemorrhagic lesions changed, with chromatolysis of some of the accumulated blood and edema formation in the affected tissue confirmed in the pathological examinations. The contusions in all pigs, at 72 hours were markedly visible on T2-weighted TSE imaging, which were characterized with well-defined hyperintense area surrounded by very high signal representative of edema (FIG. 1). The volumes of contusion were measured, and expansion of cerebral contusion was calculated and compared between two groups.

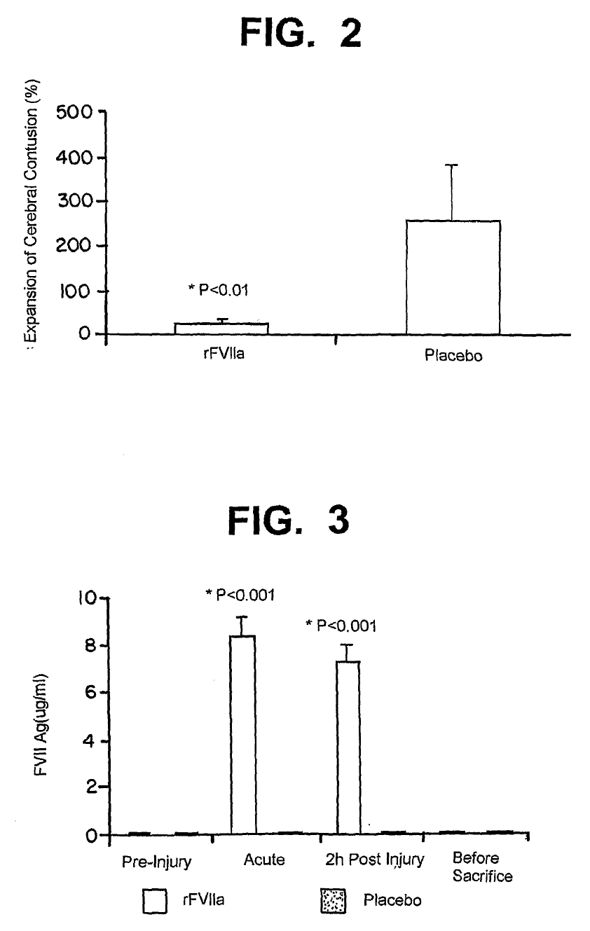

The initial volume of cerebral contusion shortly after injury was 637±142.7 mm³ in the rhFVIIa-treated animals and 684.1±129.1 mm³ in the vehicle-treated pigs with no statistically significant differences between treatment groups (p=1.0). In contrast, three days after injury, the percent expansion of contusion volume (FIG. 2) was 25.54±6.4 for the rhFVIIa-treated group and 256.7±128.7 for the vehicle-treated group (p=0.0079).

### Coagulation Parameters

In the rhFVIIa-treated group, FVII activity, measured by FVIIAg-EIA, increased significantly (P<0.05) from baseline levels for the 5 minutes post-dosing and at 2 hrs-post-injury. It declined to normal levels at three-day post-injury (FIG. 3). The observations are consistent with the half life of rhFVIIa (2.6 hr).

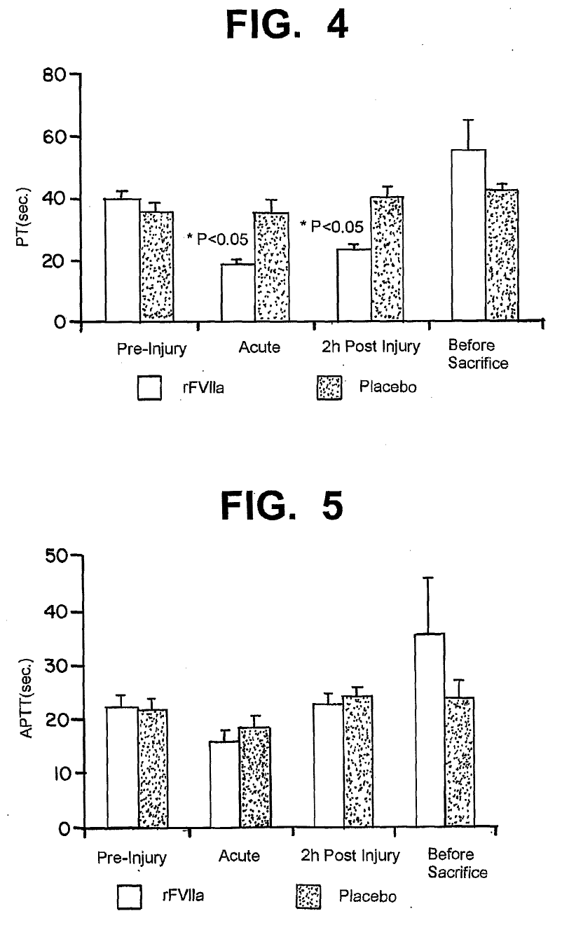

Consistent with the increase in rFVII activity, the prothrombin time (PT) decreased significantly (P<0.05) in the rhFVIIa-treatment group for blood sampled acutely and two hours after injury when compared with the vehicle-treated group. At 3 days post-injury there were no significant differences in PT between treatment groups (FIG. 4). There was no effect of rhFVIIa on APTT (FIG. 5).

### Gross Pathological Findings

The volume of cerebral contusions on gross pathology as well as microbleedings detected under the microscope were analyzed and compared with the results obtained by MRI on day 3. A significant correlation between histological findings and MRI images was demonstrated (r=0.69, p<0.005).

### Examination of Hippocampal Neurons

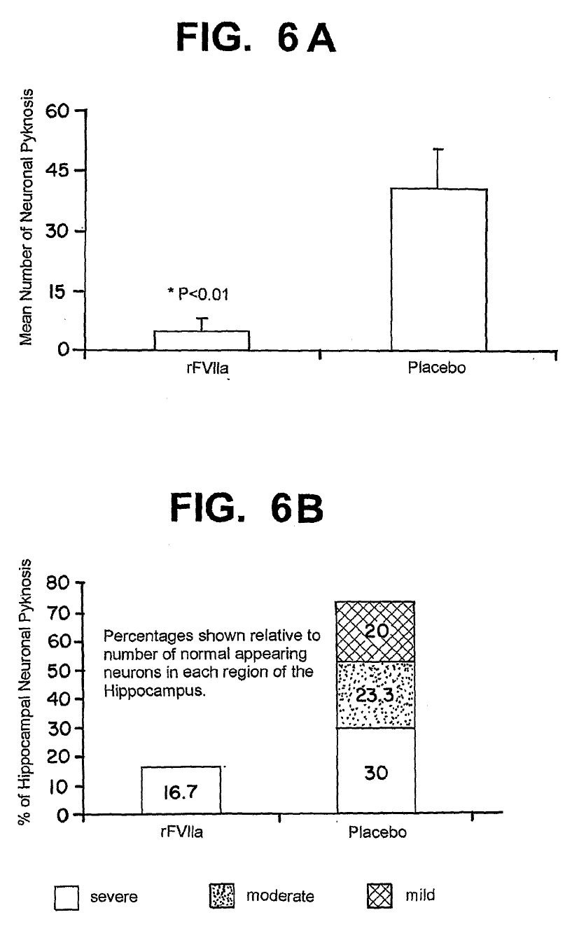

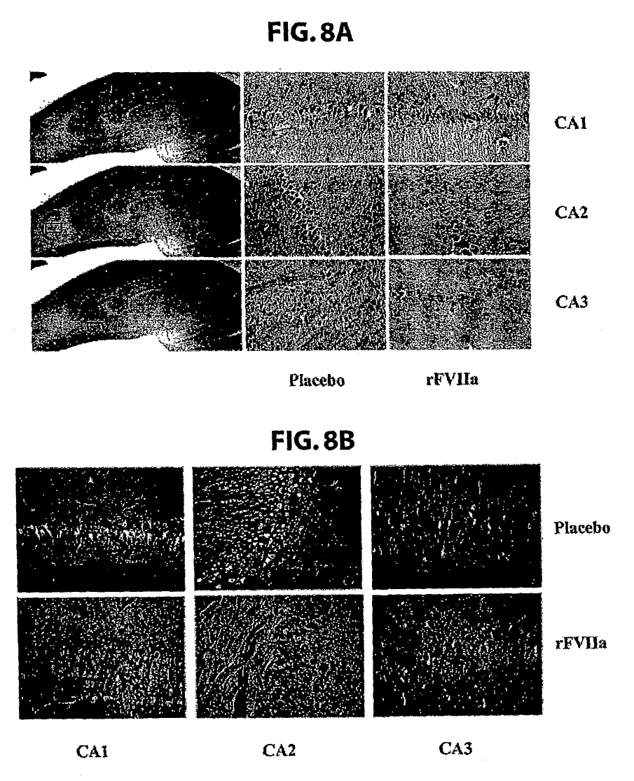

At day 3 post injury, H&E staining demonstrated pyknotic neurons (shrunken dark nuclei diagnostic for neuron death) in the CA1, CA2, and CA3 regions of bilateral hippocampus in the vehicle-treated group. Few degenerated neurons, however, were detected in these three subregions in rhFVIIa-treated pigs (representative photomicrographs of H&E(A) and Fluorojade(B) stain in three subfields of the hippocampus at three days after injury and treatment were obtained (FIG. 8); attenuation of neuronal damage following rFVIIa treatment was noted). Statistically, there was a significant reduction in neuronal pyknosis in rhFVIIa-treated pigs compared to the vehicle-treated animals (p<0.01) in the hippocampal region on both ipsilateral and contralateral to the contusion injured brain (FIGS. 6a and 6b).

### Examination of Intravascular Coagulation

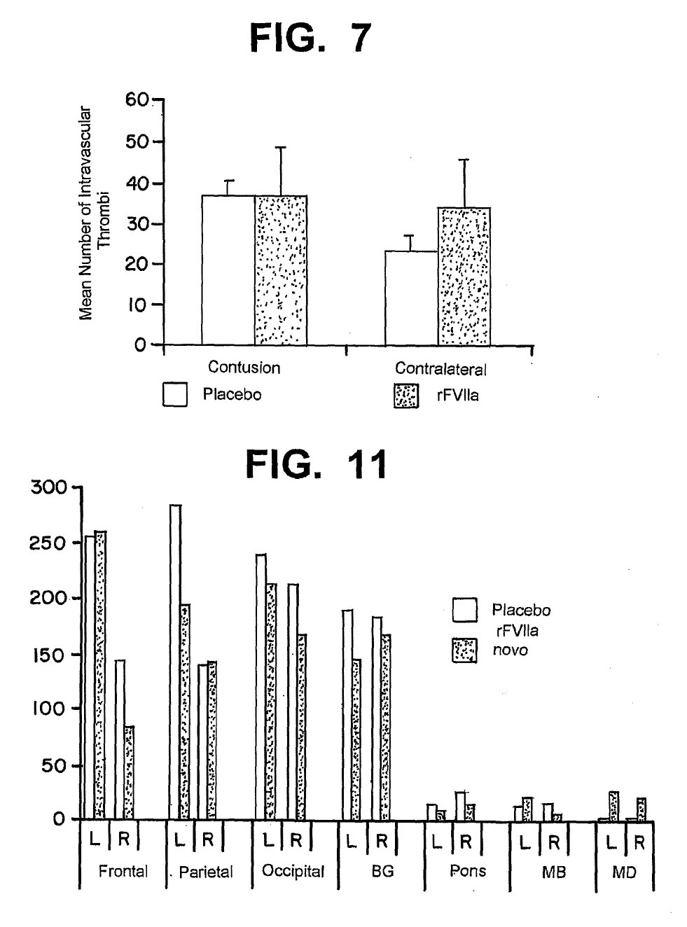

For the evaluation of intravascular coagulation in the brain, fibrin thrombi were to be detected in small and medium-sized arterioles and venules by immuno-fluorescence staining in either rhFVIIa-treated or vehicle-treated animals. In the hemisphere with cerebral contusion, there was no difference between these two groups in the severity of intravascular thrombosis (FIG. 7). In the contralateral hemisphere, it appeared that rhFVIIa-treated animals had slightly more intravascular thrombi than vehicle-treated pigs; however, this difference was not statistically significant (P=0.67).

Histological examination of lung and liver provided no evidence of intravascular thrombosis in any of the treated animals.

### DTI Analysis

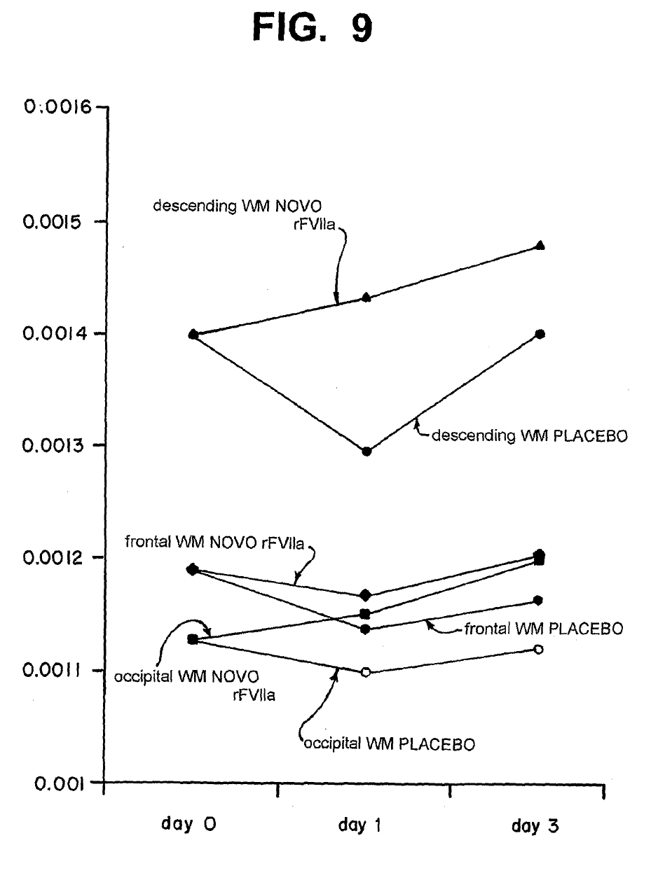

Fractional Anisotropy (FA): The descending white matter tracts in the internal capsule had significantly higher FA (P<0.05) than in the hemispheric white matter. This finding is expected as axons are packed more tightly, with fewer crossing fibers, in the descending white matter tracts as compared to the subcortical white matter in the frontal and occipital lobes. There were only minimal changes in FA at Day 1 and Day 3 as compared to normal controls, however, at Day 1, the mean FA values in the placebo treated animals were noted to be lower than those treated with rhFVIIa, and this finding was significant for the internal capsule (P≦0.05). Longitudinal Diffusion: At Day 1 we see lower longitudinal diffusion values in the placebo treated animals, as compared to rhFVIIa treated animals, in the frontal white matter (P≦0.10), occipital white matter (P≦0.15) and internal capsule (P≦0.05), which persisted in the occipital white matter (P≦0.05) (FIG. 9).

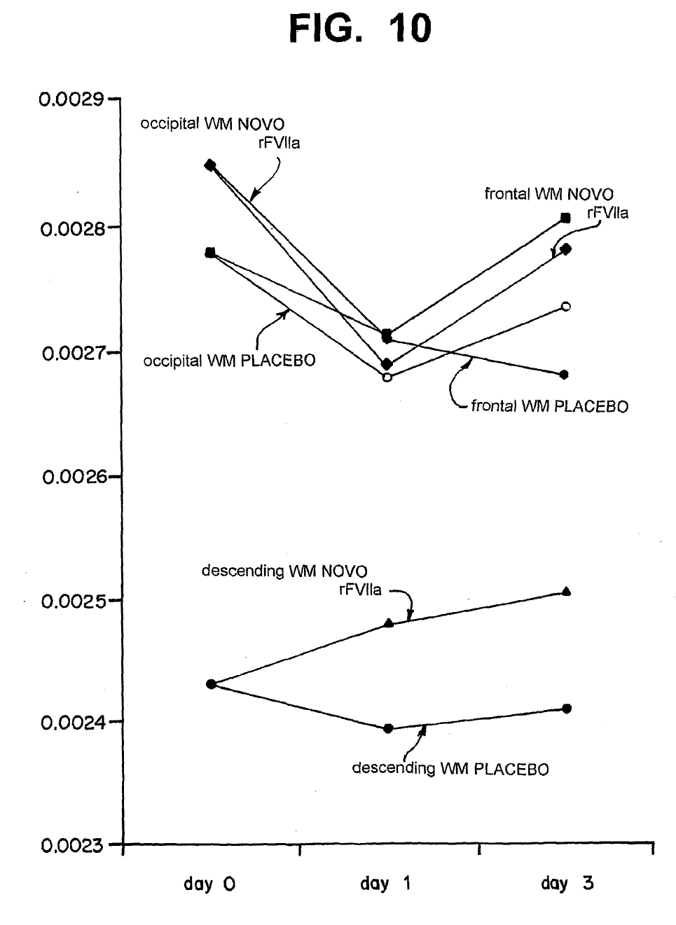

Mean Diffusion: In the hemispheric white matter, there appears to be a slight decrease in mean diffusion at Day 1, which rebounds by Day 3, with decreased values seen in the rhFVIIa group, as compared to the placebo group, in the frontal white matter and internal capsule (P≦0.15) (FIG. 10).

Overall, after injury there was a decrease in proton diffusivity in the white matter of vehicle treated animals, suggesting obstruction of axons due to a combination of axon swelling, cytoskeletal damage and disconnection. This loss of proton diffusion was consistent with histologic identification of axonal pathology in the same regions. In animals treated with rFVIIa after injury, there was a reduced loss of diffusivity compared to vehicle treated animals. In addition, regions showing reduced proton diffusivity in rFVIIa treated animals corresponded with a regionally reduced extent of axonal pathology found with histologic examination. These regions include the descending tracts in the frontal, parietal and occipital lobes and in the brain stem.

### Examination of Axonal Pathology

The distribution and severity of axonal injury in vehicle vs. rFVIIa treated animals was evaluated at 3 days post-TBI (FIG. 11). A notable and substantial decrease in the number of swollen and disconnected axons in the rFVIIa treated animals was observed, most notably in the frontal, parietal and occipital lobes and in the basal ganglia (BG) (noted by arrows).

### Discussion

This was the first study to provide evidence on the effects of rhFVIIa in a pig TBI model including both DAI and hemorrhagic cerebral contusion. Early treatment with rhFVIIa after injury significantly reduced the expansion of cerebral contusion. Cerebral contusion expansion over three days for the rhFVIIa-treated pigs was much less when compared with vehicle-treated animals. Besides limiting the growth of the cerebral contusion, rhFVIIa induced a neuroprotective effect in the hippocampus and white matter axons following injury. Specifically, hippocampal neuronal death in rhFVIIa-treated brain-injured animals was substantially reduced compared with vehicle-treated brain-injured animals. This neuroprotective effect was present equally in both hippocampi regardless of the side of contusion. Neuroprotection of axons was also demonstrated by both histopathologic analysis and DTI analysis. After injury there was a decrease in proton diffusivity in the white matter of vehicle treated animals, consistent with histologic identification of axonal pathology in the same regions. In animals treated with rFVIIa after injury, there was a reduced loss of diffusivity compared to vehicle treated animals. In addition, the same regions showing reduced proton diffusivity in rFVIIa treated animals corresponded with a reduced extent of axonal pathology found with histologic examination.

In relation to safety, no statistically significant thrombosis either in the brain or in other crucial organs (liver and lung) was found in rhFVIIa-treated pigs compared to vehicle-treated pigs. Finally, rhFVIIa also did not induce any effects on the nature of the axonal injury following injury.

Not wishing to be bound by theory, there are two components to the proposed mechanism of action by which rhFVIIa enhances hemostasis. Recombinant FVIIa binds to exposed tissue factor at the site of tissue and vascular injury, and induces the activation of factor X that causes thrombin formation. Generated thrombin activates platelets which, in turn, amplify the thrombin burst resulting in a stable clot. At high concentrations, rhFVIIa also binds to activated platelets where it can directly activate FX.

In the present study, the TBI model reproduced the most common and important pathologic features found in human TBI, including, cortical contusion, hippocampal neuron death, and diffuse axonal injury (DAI). The expansion of cortical contusion over three days following injury in rhFVIIa-treated pigs was limited significantly as compared to the vehicle-treated controls. This result confirms that rhFVIIa was able to control the growth of either spontaneous intracerebral hemorrhage or traumatic cerebral contusion. The hemorrhagic lesions, as the major pathological characteristic in traumatic contusion, are primarily induced by direct laceration of intrinsic cerebral vessels. The hemorrhage from lacerated vessels usually stops as a result of occlusion of the ruptured portion of the vessel by thrombi formation which could be enhanced by rhFVIIa according to its mechanism of action described above.

The effect of rhFVIIa on limiting contusion expansion, was also complimented by the demonstration that rhFVIIa-treatment had a significant neuroprotective effect on hippocampal neurons and white matter axons. Hippocampal neuronal death in rhFVIIa-treated animals was significantly reduced when compared with vehicle-treated animals. Likewise, neuroprotection of white matter axons in rhFVIIa-treated animals was demonstrated by both histopathologic and DTI analyses, respectively showing a reduced extend of axonal pathology and proton diffusion changes in the same brain regions. It is anticipated that by attenuating hemorrhage a reduction of numerous blood-borne neurotoxic agents from entering the brain parenchyma is obtained and/or the pro-coagulant activities of rhFVIIa can help stabilize damaged blood vessels, thereby also or alternatively reducing leakage of neurotoxic substances through the blood brain barrier (even in regions without hemorrhage). In any case, the observation that neurons in both hippocampi with spared and there was a reduction of axonal pathology throughout the white matter demonstrates that rhFVIIa treatment after TBI has a widespread neuroprotective effect far beyond regions of hemorrhage.

In the present study, no significant difference in intravascular coagulation in the hemisphere with cortical contusion was found between two groups. No intravascular thrombi were present in vital organs including liver and lung. These results support the safety profile of rhFVIIa in the setting of traumatic brain injury.

### CONCLUSION

Early treatment with rhFVIIa in the above-described TBI pig model limited expansion of a cerebral contusion without exacerbating intravascular coagulation. Recombinant human FVIIa treatment was also demonstrated to provide a neuroprotective effect on hippocampal neurons and white matter axons after TBI. There was no evidence of any significant increase in microthrombi in rhFVIIa-treated animals compared to vehicle-treated animals at time of autopsy. These data suggest a potential role for the use of rhFVIIa to treat hemorrhagic contusions in TBI patients and for the treatment of related neuropathological conditions.

All references, including publications, patent applications, and patents, cited herein are hereby incorporated by reference to the same extent as if each reference were individually and specifically indicated to be incorporated by reference and were set forth in its entirety herein (to the maximum extent permitted by law).

The use of the terms “a” and “an” and “the” and similar referents in the context of describing the invention are to be construed to cover both the singular and the plural, unless otherwise indicated herein or clearly contradicted by context.

Recitation of ranges of values herein are merely intended to serve as a shorthand method of referring individually to each separate value falling within the range, within two orders of magnitude, unless otherwise indicated herein, and each separate value is incorporated into the specification as if it were individually recited herein (e.g., a disclosure of 1-2 should be interpreted as individually disclosing 1.1, 1.2, 1.3, 1.4, 1.5, 1.6, 1.7, 1.8, 1.9, and 2.0). Unless otherwise stated, all exact values provided herein may be representative of corresponding approximate values and visa versa (e.g., all exact exemplary values provided with respect to a particular factor or measurement can be considered to also provide a corresponding approximate measurement, modified by “about,” where appropriate).

All methods described herein can be performed in any suitable order unless otherwise indicated herein or otherwise clearly contradicted by context.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

1. A method for providing neuroprotection in a gyrencephalic mammal suffering from or at substantial risk of developing a neuropathological condition not primarily induced by apoptosis comprising administering to the mammal a neuroprotective-effective dose of Factor VIa or a neuroprotective Factor VIa equivalent and assessing the neurological state of the mammal.

2. The method of claim 1, wherein the neuropathological condition is induced by an injury to the central nervous system (CNS).

3. The method of claim 2, wherein the neuropathological condition is a traumatic brain injury (TBI).

4. The method of claim 3, wherein the TBI is associated with a brain contusion and the Factor VIa or neuroprotective Factor VIa equivalent is administered in a dose and under conditions such that there is or would be an at least about 5-fold reduction in brain contusion in the mammal after occurrence of the injury.

5. The method of claim 3, wherein a significant component of the TBI is diffuse axonal injury (DAI) and the Factor VIa or neuroprotective Factor VIa equivalent is administered in a dose and under conditions such that there is or would be a detectable decrease in DAI in the mammal after occurrence of the injury.

6. The method of claim 3, wherein administration of the factor VIa or neuroprotective factor VIa equivalent leads to reduction in the number of pyknotic neurons in the hippocampus after occurrence of the injury by at least about 25%.

7. The method of claim 3, wherein administration of Factor VIa or the neuroprotective Factor VIa equivalent results in a detectable reduction in amyloid beta and/or amyloid plaques in the mammal.

8. The method of claim 7, wherein the reduction in amyloid plaques is sufficiently great to prevent the development of TBI-related Alzheimer's disease or Parkinson's disease in the mammal.

9. The method of claim 2, wherein the neuropathological condition is spinal injury.

10. The method of claim 1, wherein the neuropathological condition is drug-induced neurodegeneration, toxin-induced neurodegeneration, or surgery-related neurodegeneration.

11. The method of claim 1, wherein the neuropathological condition is caused by an infection other than HIV infection or an intracranial tumor.

12. The method of claim 1, wherein the neuropathological condition is diabetic neuropathy.

13. The method of claim 1, wherein the neuropathological condition is caused by vitamin deficiency, uremia, anoxia, chronic liver disease, lysosomal storage disease, or fragile X syndrome.

14. A method for providing neuroprotection in a mammal that is suffering from or is at substantial risk of developing a neuropathological condition that manifests as a seizure disorder or neuropsychiatric disorder comprising administering to the mammal a neuroprotective-effective dose of Factor VIa or a neuroprotective Factor VIa equivalent.

15. The method of claim 14, wherein the mammal suffers from a seizure disorder.

16. The method of claim 15, wherein the seizure disorder is epilepsy.

17. The method of claim 16, wherein the seizure disorder is status epilepticus.

18. A method for providing neuroprotection in a mammal that is suffering from or is at substantial risk of developing a neuropathological condition that manifests as a blinding eye disease, comprising administering to the mammal a neuroprotective-effective dose of Factor VIa or a neuroprotective Factor VIa equivalent.

19. The method of claim 18, wherein the disease is selected from the group consisting of macular degeneration, retinitis pigmentosa, and glaucoma.

20. A method of providing neuroprotection in a mammal suffering from or being at substantial risk of developing a white matter disease-associated or injury-associated neuropathological condition comprising administering to the mammal a neuroprotective-effective dose of Factor VIa or a neuroprotective Factor VIa equivalent, with the proviso that the neuropathological condition is not Parkinson's disease, Alzheimer's disease, amyotrophic lateral sclerosis (ALS), denervation atrophy, otosclerosis, stroke, dementia, multiple sclerosis, Huntington's disease, or AIDS-associated encephalopathy.

21-62. (canceled)
